(19) United States
(12) Patent Application Publication
(10) Pub. No.: US 2023/0266325 A1
(43) Pub. Date: Aug. 24, 2023

(54) METHODS FOR DETECTING LUNG CANCER

(71) Applicant: LungLife AI, Inc., Thousand Oaks, CA (US)

(72) Inventors: Paul PAGANO, Moorpark, CA (US); Daniel GRAMAJO-LEVENTON, North Hills, CA (US); Rebecca REED, Sherman Oaks, CA (US); Shahram TAHVILIAN, Tarzana, CA (US); Lara BADEN, Woodland Hills, CA (US); Ashley BROWN, Simi Valley, CA (US)

(21) Appl. No.: 18/011,724

(22) PCT Filed: Jun. 30, 2021
(86) PCT No.: PCT/US2021/039915

§ 371 Date: Dec. 20, 2022

Related U.S. Application Data
(60) Provisional application No. 63/046,456, filed on Jun. 30, 2020.

(51) Int. Cl. G01N 33/574; C12Q 1/6886

(52) U.S. Cl. CPC G01N 33/57423; C12Q 1/6886; C12Q 2600/118; G01N 2333/70592

(57) ABSTRACT

The present disclosure provides a method for of determining the level of circulating tumor cells (CTCs) in a sample having blood cells from a patient comprising obtaining a test sample from a human subject; enriching circulating tumor cells (CTC); hybridizing the enriched cells in the sample with labeled nucleic acid probes that hybridize to regions of chromosomal DNA; evaluating the signal pattern for the selected cells by detecting fluorescence in situ hybridization from cells; detecting CTCs based on the pattern of hybridization to the labeled nucleic acid probes to said selected cells; and identifying the subject at risk for the development of lung cancer when the number of CTC per sample is above a predetermined cutoff value.--

### CROSS REFERENCE TO RELATED APPLICATIONS

This application claims priority to U.S. Provisional Application No. 63/046,456, filed Jun. 30, 2020, which is incorporated by reference herein in its entirety for all purposes.

### BACKGROUND OF THE INVENTION

Computed tomography is the standard method by which pulmonary nodules are detected whether incidentally or as part of a lung cancer screening program. Radiological characteristics and clinical risk assessment performed guides clinicians when a biopsy is indicated. It is estimated that greater than 40% of biopsies of suspicious pulmonary nodules are not lung cancer and are therefore unnecessary. Transthoracic biopsies often lead to complications including infection, pneumothorax, hemorrhage and even death.

The disclosure provides a 4-color fluorescence in-situ hybridization assay detecting early circulating tumor cells from peripheral blood draw. A non-limiting example of the 4-color fluorescence in-situ hybridization assay is a LungLB™ assay. In some aspects, the 4-color fluorescence in-situ hybridization assay aids the clinical assessment of patients with indeterminate nodules suspicious for lung cancer. In some aspects, the assay is based on the observation that the metastatic process is active early in lung cancer pathogenesis.

A blinded study of 46 subjects receiving a LungLB™ assay blood draw concurrent with nodule biopsy (n=31 malignant, n=15 with benign nodules) was conducted. Using receiver operating characteristic curve (ROC) analysis, a sensitivity of 81% and specificity of 87% with an area under the curve (AUC) of 0.823 was achieved. Clinical factors commonly used in malignancy prediction models were also assessed and were found to be non-informative, suggesting data reflect “real world” scenarios and are not biased by available clinical factors.

Early clinical performance of the LungLB™ assay suggests it may be useful as an adjunct to the clinical assessment of indeterminate lung nodules. Had LungLB™ been used in patient with benign lesions, 13 of 15 subjects in this study may have been spared from biopsy. Large-scale validation and utility studies are warranted.

There has been a long-felt but unmet need in the art for methods of detecting lung cancers in humans. The estimated number of new lung cancer cases exceeded 230,000 in 2018, and the five-year survival rate has marginally increased from 11.4% in 1975 to 17.5% in 2013 (Siegel RL, et al., 2018, CA Cancer J Clin.;68(1):7-30). This is in part due to the lack of early detection and that early stage disease is typically asymptomatic. As such, the majority of cases are caught in the later stages of disease with detectable metastatic disease. The ability to identify lung cancer at earlier stages would have a significant impact on the overall outcome of lung cancer patients.

Low-dose computed tomography (LDCT) is the standard for lung cancer screening and the National Lung Screening Trial showed a 20% reduction in lung cancer-specific mortality (Aberle D.R., Adams A.M. et al. N Engl J Med. (2011) 365(5):395-409). While highly sensitive, LDCT suffers from low specificity and a high rate of false positives, even when incorporating current LungRADS criteria (Pinsky P.F., Gierada D.S. et al. (2015) Ann Intern Med. 162(7):485-91). It is estimated that greater than 40% of biopsies of indeterminate pulmonary nodules identified by CT scan are negative for lung cancer (Lokhandwala T, Bittoni M.A. et al. (2017) Clin Lung Cancer. 18(1):e27-e34. doi: 10.1016/j.cllc.2016.07.006. Epub 2016 Jul 21), and as reported nearly 20% of biopsy patients are subject to adverse events.

Using blood for cancer diagnostics is a promising approach given the specimen can be obtained inexpensively and often less invasively than tissue biopsy. Blood-based biomarkers for cancer detection have attracted much research interest, especially in lung cancer where biopsy is challenging (Zugazagoitia, Ramos et al. (2019). Ann Oncol 30(2): 290-296). Whole blood is a complex mixture that includes plasma and cell-based compartments, each of which contains unique biomarkers that are often complimentary (Hodara, Morrison et al. (2019). JCIInsight 4(5)). Plasma contains circulating-free DNA (cfDNA, from normal and tumor [ctDNA] tissues), exosome-containing RNA, and various proteinaceous components. The cellular compartment contains normal blood cells and tumor-derived cells (circulating tumor cells, CTC). One of the main advantages for using blood as opposed to traditional biopsy is that the specimen is not restricted to a single tumor site but rather allows a more complete sampling of the entire tumor. In particular, a CTC-based assay has the ability to detect cells that have entered the metastatic cascade, the process behind >90% of cancer-related mortality (Mehlen, Puisieux et al. (2006) Nat Rev Cancer 6(6):449-58).

Emerging technologies for early detection of lung cancer measure circulating tumor DNA (ctDNA), RNA, or proteins (Seijo et al. (2019) J Thorac Oncol 14(3): 343-357). However, these technologies are unlikely to constitute accurate early detection methods because they rely upon pathophysiological changes associated with later-stage disease (namely high tumor burden) and have biological and technical challenges that may preclude their use in detection of early cancer. While often associated with later-stage disease in many cancer types, direct measurement of circulating tumor cells (CTC) is perhaps the most promising emergent technology that provides an astute means for the detection of early lung cancer. The very early appearance of metastatic behavior in model systems and clinical lung cancers (Pagano, Tran et al. (2017) Cancer Prev Res (Phila) 10(9): 514-524 and Tanaka, Yoneda et al. (2009) Clin Cancer Res 15(22):6980-6) describes a fundamental difference in the biology of lung cancer compared to malignancies in other tissues, which can be leveraged in the early detection setting through the use of CTC-based assays. Patients diagnosed with lung cancer who are eligible for surgery with curative intent often recur and most frequently within the first two years following resection (Lou, Camelia et al. (2014) Ann Thorac Surg 95(5): 1755-1761). Depending on stage, recurrence rates range from 30-75%, the majority of which are distant (Sugimura, Nichols et al. (2007) Ann Thorac Surg 83(2):409-17). This suggests that micro-metastatic disease is present at the time of surgery, at an early stage of cancer, but below the level of detection of imaging.

Previous studies demonstrated that CTC can be identified in patients diagnosed with stage I lung cancer (Tanaka, Yoneda et al. (2009) Clin Cancer Res 15(22):6980-6, Chemi F, Rothwell DG et al.(2019) NatMed. 25(10):1534-1539), as well as those with Chronic obstructive pulmonary disease (COPD) at high-risk for lung cancer years before frank malignancy is observed radiographically (Ilie, Hofman, et al. (2014) PLoS One 9(10):e111597). The means by which a cell recovered from blood is identified as a tumor cell is critical and often times the legacy definition of cytokeratin positive/CD45 negative is insufficient. Katz et al describe a method that uses fluorescence in situ hybridization (FISH) on tumor cells enriched from the peripheral blood of patients with indeterminate pulmonary nodules for detection of copy number variation (Katz, He et al. (2010) Clin Cancer Res 16(15): 3976-3987), which is the basis of the 4-color fluorescence in-situ hybridization LungLB™ assay described herein. As FISH generally is a highly specific assay and chromosome instability is a hallmark of cancer, this method of CTC identification has advantages over commonly used protein markers such as cytokeratin and CD45 which are variably expressed within individual and across lung cancer patients.

Herein the development of a liquid biopsy assay analyzing CTC using FISH and its clinical performance is reported. The scope of this study is to evaluate concordance of a lung nodule biopsy outcome with the 4-color fluorescence in-situ hybridization LungLB™ assay test results within patients with suspicious indeterminate pulmonary nodules identified incidentally or through a lung cancer screening program.

### SUMMARY

The disclosure provides a method for identifying a subject at risk for the development of lung cancer comprising: (a) obtaining a test sample from a human subject; (b) performing a circulating tumor cell (CTC) enrichment step comprising: (i) removing plasma from the sample, (ii) removing erythrocytes from the sample, (iii) contacting the sample with at least one biotinylated affinity agent that binds a cell surface marker, and (iv) contacting the sample with streptavidin-coated magnetic particles and depleting cells from the sample that express the cell surface marker; (c) hybridizing the enriched cells in the sample with labeled nucleic acid probes that hybridize to regions of chromosomal DNA; (d) evaluating the signal pattern for the selected cells by detecting fluorescence in situ hybridization from cells; (e) detecting CTCs based on the pattern of hybridization to the labeled nucleic acid probes to said selected cells; and (f) identifying the subject at risk for the development of lung cancer when the number of CTC per sample is above a predetermined cutoff value.

In some aspects, the test sample is blood. In some aspects, the erythrocytes are removed by cell lysis. In some aspects, the cell lysis is performed by an ammonium chloride lysis buffer.

In some aspects, the plasma is removed by centrifugation.

In some aspects, the cell surface marker is selected from CD66b, CD14, CD3, CD4, CD8, CD17, CD56, CD19, CD20, CD25, IgM, or IgD. In some aspects, the cell surface marker is selected from CD66b, CD3 or CD14. In some aspects, the cell surface marker comprises CD66b and CD14. In some aspects, the cell surface marker comprises CD66b, CD14 and CD3. In some aspects, the cell surface marker comprises CD66b, CD14, CD3, and CD56. In some aspects, the cell surface marker comprises CD66b, CD14, CD3, and CD19. In some aspects, the cell surface marker comprises CD66b, CD14, CD3, CD56 and CD19.

In some aspects, the at least one biotinylated affinity agent comprises an anti-CD66b, anti-CD3, anti-CD56, anti-CD19 or anti-CD14 antibody. In some aspects, the at least one biotinylated affinity agent comprises an anti-CD66b antibody and an anti-CD14 antibody. In some aspects, the at least one biotinylated affinity agent comprises an anti-CD66b antibody, an anti-CD14 antibody, and an anti-CD3 antibody. In some aspects, the at least one biotinylated affinity agent comprises an anti-CD66b antibody, an anti-CD14 antibody, an anti-CD3 antibody, and an anti-CD56 antibody. In some aspects, the at least one biotinylated affinity agent comprises an anti-CD66b antibody, an anti-CD14 antibody, an anti-CD3 antibody, and an anti-CD19 antibody. In some aspects, the at least one biotinylated affinity agent comprises an anti-CD66b antibody, an anti-CD14 antibody, an anti-CD3 antibody, an anti-CD56 antibody, and an anti-CD19 antibody.

In some aspects, the depleted cells are neutrophils, monocytes, or lymphocytes. In some aspects, the depleted cells are neutrophils and monocytes.

In some aspects, the CTC enrichment step further comprises: (i) contacting the sample with at least one additional biotinylated affinity agent that binds a cell surface marker, and (iv) contacting the sample with streptavidin-coated magnetic particles and collecting cells that express the cell surface marker.

In some aspects, the cell surface marker comprises at least one of CD19, CD20, IgM, or IgD. In some aspects, the at least one additional biotinylated affinity agent comprises at least one of an anti-CD19 antibody, an anti-CD20 antibody, an anti-IgM antibody, or an anti-igD antibody.

In some aspects, the collected cells comprise lymphocytes. In some aspects, the lymphocytes are B-cells.

In some aspects, the labeled nucleic acid probes comprise 3p22.1, 10q22.3, chromosome 10 centromeric (cep10), and 3q29.

In some aspects, the subject at risk has indeterminate pulmonary nodules.

In some aspects, a CTC is identified when the hybridization pattern of the nucleic acid probes depicts a gain of two or more chromosomal regions in a cell.

In some aspects, a CTC is identified when the hybridization pattern of the nucleic acid probes depicts a loss of two or more chromosomal regions in a cell.

In some aspects, a CTC count greater than 1 CTC/10,000 cells represents a risk of lung cancer. In some aspects, a CTC count greater than 2 CTC/10,000 cells represents a risk of lung cancer. In some aspects, a CTC count greater than 2.5 CTC/10,000 cells represents a risk of lung cancer. In some aspects, a CTC count greater than 5 CTC/10,000 cells represents a risk of lung cancer. In some aspects, a CTC count greater than 10 CTC/10,000 cells represents a risk of lung cancer. In some aspects, a CTC count greater than 20 CTC/10,000 cells represents a risk of lung cancer. In some aspects, the subject with a CTC count greater than 5 CTC/10,000 cells is referred for surgical resection of the nodule.

In some aspects, the labeled nucleic acid probes for 3p22.1 is an RPL14, CD39L3, PMGM, or GC20 probe. In some aspects, the labeled nucleic acid probes for 10q22.3 is a surfactant protein A1 or surfactant protein A2 probe.

The disclosure provides a method for identifying a subject at risk for the development of lung cancer comprising: (a) obtaining a test sample from a human subject; (b) performing a circulating tumor cell (CTC) enrichment step comprising: (i) removing plasma from the sample, (ii) removing erythrocytes from the sample, (iii) contacting the sample with at least one biotinylated affinity agent that binds a cell surface marker, and (iv) contacting the sample with streptavidin-coated magnetic particles and collecting cells from the sample that express the cell surface marker; (c) hybridizing the enriched cells in the sample with labeled nucleic acid probes that hybridize to regions of chromosomal DNA; (d) evaluating the signal pattern for the selected cells by detecting fluorescence in situ hybridization from cells; (e) detecting CTCs based on the pattern of hybridization to the labeled nucleic acid probes to said selected cells; and (f) identifying the subject at risk for the development of lung cancer when the number of CTC per sample is above a predetermined cutoff value.

In some aspects, the cell surface marker is selected from CD66b, CD14, CD3, CD4, CD8, CD17, CD56, CD19, CD20, CD25, IgM, or IgD.

In some aspects, the cell surface marker is a B-cell specific cell surface marker. In some aspects, the B-cell specific cell surface marker is CD19, CD20, IgM, or IgD. In some aspects, the at least one biotinylated affinity agent comprises an anti-CD19 antibody, an anti-CD20 antibody, an anti-IgM antibody, or an anti-IgD antibody.

The disclosure provides a method of evaluating cancer in a subject comprising determining the level of circulating tumor cells (CTCs) in a sample containing blood cells from the patient by the method of any one of the preceding claims, wherein a higher level of CTCs in the sample, as compared to a control or predetermined number of CTCs from a non-aggressive form of cancer, is indicative of an aggressive form of cancer and/or a poor cancer prognosis.

The disclosure provides a method of staging cancer in a subject comprising determining circulating tumor cells (CTC) in a sample containing blood cells from the subject by the method of any one of the preceding claims, wherein a higher level of CTCs in the sample as compared to a predetermined control for a given stage is indicative of a more advanced stage of cancer, and a lower level of CTCs in the sample as compared to a control for a given stage is indicative of a less advanced stage of cancer.

### BRIEF DESCRIPTION OF THE DRAWINGS

FIGS. 1 is a series of flow cytometry dot plots depicting erythrocyte and granulocyte depletion by ficoll versus erythrocyte lysis with magnetic depletion. FIG. 1A depicts lysed blood without cell enrichment and shows a high percentage of granulocytes and monocytes and low lymphocytes. FIG. 1B depicts the result of density separation, which removes most granulocytes but lymphocyte purity is still insufficient at <80%. FIG. 1C depicts the result of magnetic depletion using CD66b and CD14 antibodies, and displays the highest percentage of lymphocytes (>90%) suitable for CTC enrichment.

FIG. 2 is an image depicting copy number variation observed in a 4-color fluorescence in-situ hybridization (LungLB™) assay.

FIG. 3 is a graph depicting flow cytometry data showing higher monocytes and granulocytes in false negative samples.

FIG. 4 is a graph depicting total cell count data showing fewer cells in false negative samples.

FIG. 5 is a graph depicting that average CTC count is doubled when CD14+CD66b are used in depletion compared to CD66b alone.

FIG. 6 is a graph depicting stability of cells following cryopreservation at 0.5, 1, and 3 months for depletion efficiency and FISH.

FIG. 7 is a set of images depicting fresh cells and cryopreserved cells following 3 months of cryopreservation.

FIG. 8 is a scatter plot showing the count distribution in healthy donor blood. The dotted line represents the threshold determined using ROC analysis on clinical specimens.

FIG. 9 is a graph depicting linearity of the 4-color fluorescence in-situ hybridization (LungLB™) assay using A549 cells spiked into healthy blood.

FIG. 10 is a graph depicting a receiver operator characteristics curve of 4-color fluorescence in-situ hybridization (LungLB™) assay in patients with indeterminate pulmonary nodules.

FIG. 11 is a series of images depicting example CTC in a patient with benign biopsy but positive 4-color fluorescence in-situ hybridization (LungLB™) assay test.

FIGS. 12A-12C is a series of graphs depicting changes in granulocyte size upon exposure to cell lysis buffers with varying sodium bicarbonate concentrations.

FIG. 13A is a graph depicting CTC ratio / 10,000 cells following depletion using CD66b, CD14 antibodies or CD66b, CD14, and CD3 antibodies in positive and negative samples.

FIG. 13B is a table depicting total cell count in 2 or 3 antibody depletion samples along with their CTC ratio (CTCs/10,000 cells) and their identification following the assay (true positive, true negative, false positive).

FIG. 14A is an immunofluorescence image of CTCs visualized using DAPI stain. Target cell 1606 is boxed and identified on the image.

FIG. 14B is an immunofluorescence image of CTCs visualized using CD45-FITC stain. Target cell 1606 is boxed and identified on the image as being CD45 negative (no green fluorescence).

FIG. 14C is an image of target cell 1606 following LungLB assay and depicts a pattern of 4R/2Gd/4Gr/2Aq. R = Red; 3p22.1, Gd = gold; 10q22.3, Gr = green; 3q29, and Aq = aqua; 10 centromeric.

FIG. 15A is a series of photos depicting Target cell 4255 stained with DAPI (left image), CD45-FITC (center image), and LungLB assay images (Right images). Target cell 4255 is CD45 negative and identified as 2R/4Gd/2Gr/4Aq. R = Red, Gd = gold, Gr = green, and Aq = aqua.

FIG. 15B is a series of photos depicting Target cell 4259 stained with DAPI (left image), CD45-FITC (center image), and LungLB assay images (Right image). Target cell 4259 is CD45 positive and identified as 3R/2Gd/3Gr/2Aq. R = Red; 3p22.1, Gd = gold; 10q22.3, Gr = green; 3q29, and Aq = aqua; 10 centromeric.

FIGS. 16 is a series of photos depicting Target cell 16270 stained with DAPI (FIG. 16A), CD45-FITC (FIG. 16B), and LungLB assay images (FIG. 16C). Target cell 16270 is CD45 positive and identified as 3R/2Gd/3Gr/2Aq. R = Red; 3p22.1, Gd = gold; 10q22.3, Gr = green; 3q29, and Aq = aqua; 10 centromeric.

FIG. 17A is a flow cytometry dot plot depicting identification of CD19+ or CD19- cells using an immunofluorescent anti-CD19 antibody. CD19+ cells are B-cells.

FIG. 17B is a flow cytometry dot plot depicting identification of CD56+ or CD56- cells using an immunofluorescent anti-CD56 antibody. CD56+ cells are Natural Killer (NK) cells.

### DETAILED DESCRIPTION

### Methods of the Disclosure

In some aspects, the present disclosure provides methods for identifying a subject at risk for the development of cancer. In some aspects, the present disclosure provides methods of detecting cancer in a subject. In some aspects, the subject at risk has one or more indeterminate pulmonary nodules.

In some aspects, the present disclosure provides methods for identifying a subject at risk for the development of lung cancer. In some aspects, the present disclosure provides methods of detecting lung cancer in a subject.

In some aspects, the present disclosure provides methods for identifying a subject at risk for the development of cancer comprising: obtaining a test sample from a human subject; performing a circulating tumor cell (CTC) enrichment step comprising: removing plasma from the sample, removing erythrocytes from the sample, contacting the sample with at least one affinity agent that binds a cell surface marker, and depleting cells from the sample that express the cell surface marker; hybridizing the enriched cells in the sample with labeled nucleic acid probes; evaluating the signal pattern for the selected cells by detecting fluorescence in situ hybridization from cells; detecting CTCs based on the pattern of hybridization to the labeled nucleic acid probes to said selected cells; and identifying the subject at risk for the development of lung cancer when the number of CTC per sample is above a predetermined cutoff value.

In some aspects, the present disclosure provides methods for identifying a subject at risk for the development of cancer comprising: obtaining a test sample from a human subject; performing a circulating tumor cell (CTC) enrichment step comprising: removing plasma from the sample, removing erythrocytes from the sample, contacting the sample with at least one biotinylated affinity agent that binds a cell surface marker, and contacting the sample with streptavidin-coated magnetic particles and depleting cells from the sample that express the cell surface marker; hybridizing the enriched cells in the sample with labeled nucleic acid probes; evaluating the signal pattern for the selected cells by detecting fluorescence in situ hybridization from cells; detecting CTCs based on the pattern of hybridization to all four labeled nucleic acid probes to said selected cells; and identifying the subject at risk for the development of lung cancer when the number of CTC per sample is above a predetermined cutoff value.

In some aspects, the subject at risk for the development of cancer is at risk for developing cancers of lung, breast, colon, prostate, pancreas, esophagus, all gastro-intestinal tumors, urogenital tumors, kidney cancers, melanomas, endocrine tumors, sarcomas, etc. In some aspects, the subject at risk for the development of lung cancer.

In some aspects, the test sample comprises blood cells. In some aspects, the test sample comprises saliva, peripheral blood cells, bone marrow, or stem cells isolated from blood or bone marrow. In some aspects, the test sample is peripheral blood.

In some aspects, the peripheral blood is obtained from the subject by a peripheral blood draw.

### Circulating Tumor Cell Enrichment

The present disclosure provides an improved and superior method of enriching and isolating circulating tumor cells (CTC) from a test sample. In some aspects, the present disclosure provides a method of performing a circulating tumor cell (CTC) enrichment step comprising: removing plasma from the sample, removing erythrocytes from the sample, contacting the sample with at least one biotinylated affinity agent that binds a cell surface marker, and contacting the sample with streptavidin-coated magnetic particles and depleting cells from the sample that express the cell surface marker.

In some aspects, the CTCs are enriched from a test sample wherein the test sample is whole blood. In some aspects, the sample is fresh blood. In some aspects, the sample is fixed blood. In some aspects, fixed blood is blood that is stabilized using chemicals that cross-link proteins and DNA such that normal clotting and degradation processes are significantly slowed or stopped.

In some aspects, plasma is removed from the sample. In some aspects, plasma is removed from the sample by centrifugation. In some aspects, the sample is centrifuged for at least 1 min, at least 2 min, at least 3 min, at least 4 min, at least 5 min, at least 6 min, at least 7 min, at least 8 min, at least 9 min, at least 10 min, at least 11 min, at least 12 min, at least 13 min, at least 14 min, at least 15 min, or at least 20 min. In some aspects, the sample is centrifuged for 10 min. In some aspects, the sample is centrifuged at 100 x g, 200 x g, 300 x g, 400 x g, 500 x g, 600 x g, 700 x g, 800 x g, 900 x g, or 1000 x g. In some aspects, the sample is centrifuged at 700 x g.

In some aspects, following centrifugation, the plasma is removed from the sample and stored at -80° C.

In some aspects, removal of neutrophils, monocytes, and granulocytes reduces the rate of false negative samples as analyzed by FISH.

In some aspects, erythrocytes are removed from the sample. In some aspects, erythrocytes are removed by cell lysis. In some aspects, the sample is contacted with an erythrocyte lysis buffer. In some aspects, the erythrocyte lysis buffer is an ammonium chloride lysis buffer. In some aspects, the erythrocyte lysis buffer contains ammonium chloride. In some aspects, the erythrocyte lysis buffer contains sodium bicarbonate. In some aspects, the erythrocyte lysis buffer contains ethylenediaminetetraacetic acid (EDTA). In some aspects, the erythrocyte lysis buffer contains ammonium chloride (8.29 grams), sodium bicarbonate (0.2 grams), Ethylenediaminetetraacetic acid (1.1 grams) and water (90.494 milliliters). In some aspects, the erythrocyte lysis buffer contains ammonium chloride at a concentration of 0.01 M to 5 M, 0.1 M to 4 M, 0.5 M to 3 M, or 1 M to 2 M. In some aspects, the erythrocyte lysis buffer contains ammonium chloride at a concentration of 1.0 M, 1.1 M, 1.2 M, 1.3 M, 1.4 M, 1.5 M, 1.55 M, 1.6 M, 1.7 M, 1.8 M, 1.9 M, or 2 M. In some aspects, the erythrocyte lysis buffer contains sodium bicarbonate at a concentration of 1 mM to 200 mM, 5 mM to 150 mM, 15 mM to 100 mM, or 20 mM to 40 mM. In some aspects, the erythrocyte lysis buffer contains sodium bicarbonate at a concentration of 20 mM, 21 mM, 22 mM, 23 mM, 24 mM, 25 mM, 26 mM, 27 mM, 28 mM, 29 mM, or 30 mM. In some aspects, the erythrocyte lysis buffer contains Ethylenediaminetetraacetic acid at a concentration of 1 mM to 200 mM, 5 mM to 150 mM, 15 mM to 100 mM, or 25 mM to 45 mM. In some aspects, the erythrocyte lysis buffer contains Ethylenediaminetetraacetic acid at a concentration of 30 mM, 31 mM, 32 mM, 33 mM, 34 mM, 35 mM, 36 mM, 37 mM, 37.6 mM, 38 mM, 39 mM, 40 mM, 41 mM, 42 mM, 43 mM, 44 mM, or 45 mM.

In some aspects, the sodium bicarbonate concentration is different for fresh blood and fixed blood samples. In some aspects, different sodium bicarbonate concentrations alter the number of granulocytes that change in size and granularity. In fixed blood, the widely-used bicarbonate concentration results in a left-shift (size reduction) of granulocytes. In some aspects, increased sodium bicarbonate concentration exacerbates the observation. In some aspects, lower sodium bicarbonate concentration rescues the phenotype (granulocytes keep a normal size).

In some aspects, following erythrocyte removal, cells are further removed from the sample using magnetic depletion. In some aspects, the sample is contacted with at least one biotinylated affinity agent. In some aspects, the biotinylated affinity agent binds a cell surface marker. In some aspects, the cell surface marker is specific for a cell type. In some aspects, the cell type is a neutrophil, monocyte, plasma cell or lymphocyte. In some aspects, the cell type is a neutrophil or monocyte. In some aspects, the lymphocyte is a B-cell and subpopulations thereof, a natural killer (NK) cell and subpopulations thereof, or a T-cell and subpopulations thereof. In some aspects, the B-cell is a naïve B-cell or a mature B-cell. In some aspects, the T-cell is a T-helper cell, a cytotoxic T-cell, or regulatory T-Cells. In some aspects, the cell surface marker is CD66b, CD14, CD3, CD4, CD8, CD17, CD56, CD19, CD20, CD25, IgM, or IgD. In some aspects the cell surface marker is CD66b or CD14. In some aspects, the neutrophil cell surface marker is CD66b. In some aspects, the monocyte cell surface marker is CD14. In some aspects, CD56 is a natural killer cell surface marker. In some aspects, CD19. CD20, IgM, and IgD are B-cell surface markers.

In some aspects, the biotinylated affinity agent is an anti-CD66b antibody. In some aspects, the biotinylated affinity agent is an anti-CD14 antibody. In some aspects, the biotinylated affinity agent is an anti-CD3 antibody. In some aspects, the biotinylated affinity agent is an anti-CD4 antibody. In some aspects, the biotinylated affinity agent is an anti-CD8 antibody. In some aspects, the biotinylated affinity agent is an anti-CD17 antibody. In some aspects, the biotinylated affinity agent is an anti-CD56 antibody. In some aspects, the biotinylated affinity agent is an anti-CD19 antibody. In some aspects, the biotinylated affinity agent is an anti-CD20 antibody. In some aspects, the biotinylated affinity agent is an anti-CD25 antibody. In some aspects, the biotinylated affinity agent is an anti-IgM antibody. In some aspects, the biotinylated affinity agent is an anti-IgD antibody.

In some aspects, combinations of biotinylated affinity agents are used. In some aspects, the sample is contacted with at least two, at least three, at least four, at least five, at least six, at least seven, at least eight, at least nine, or at least ten biotinylated affinity agents. In some aspects, the sample is contacted with at least two biotinylated affinity agents. In some aspects, the sample is contacted with at least three biotinylated affinity agents. In some aspects, the sample is contacted with at least four biotinylated affinity agents. In some aspects, the sample is contacted with at least five biotinylated affinity agents. In some aspects, the sample is contacted with an anti-CD66b antibody and an anti-CD14 antibody. In some aspects, the sample is contacted with an anti-CD66b antibody, an anti-CD14 antibody, and an anti-CD13 antibody. In some aspects, the sample is contacted with an anti-CD66b antibody, an anti-CD14 antibody, an anti-CD13 antibody, and an anti-CD56 antibody. In some aspects, the sample is contacted with an anti-CD66b antibody, an anti-CD 14 antibody, an anti-CD 13 antibody, and an anti-CD 19 antibody. In some aspects, the sample is contacted with an anti-CD66b antibody, an anti-CD14 antibody, an anti-CD13 antibody, an anti-CD56 antibody, and an anti-CD19 antibody.

In some aspects, following contacting the sample with biotinylated affinity agents, the sample is contacted with streptavidin-coated magnetic particles. In some aspects, following incubation with the streptavidin-coated magnetic particles, the sample is exposed to a magnet to magnetically separate the cells expressing the targeted cell surface markers from the sample.

### Affinity Agents

In some aspects, affinity agents of the disclosure are biotinylated affinity agents. In some aspects, streptavidin-coated particles are used to bind biotinylated affinity agents and deplete and or harvest cells bound to the biotinylated affinity agent specific to a particular cell surface marker. In some aspects, affinity agents of the disclosure are directly conjugated to magnetic particles. In some aspects, affinity agents of the disclosure are Anti-Phycoerythrin (PE) MicroBeads. In some aspects, anti-PE microbeads are used for the indirect magnetic labeling and separation of cells with a PE-conjugated primary antibody. In some aspects, affinity agents of the disclosure are digoxigenin (DIG) conjugated antibodies and anti-DIG magnetic beads/particles are used in methods of the disclosure.

### Enrichment of CTCs Via Positive Selection

In some aspects, the CTC enrichment step further comprises: contacting the sample with at least one additional biotinylated affinity agent that binds a cell surface marker, contacting the sample with streptavidin-coated magnetic particles and collecting cells that express the cell surface marker. In some aspects, the collected cells are then utilized in the FISH assays described herein. In some aspects, the cell surface marker is CD66b, CD14, CD3, CD4, CD8, CD17, CD56, CD19, CD20, CD25, IgM, or IgD. In some aspects, the cell surface marker is a B-cell specific marker that comprises CD19, CD20, IgM, or IgD. In some aspects, the cell surface marker is CD66b, CD14, CD3, CD4, CD8, CD17, CD56, CD19, CD20, CD25, IgM, or IgD. In some aspects, the at least one additional biotinylated affinity agent comprises an anti-CD 19 antibody, an anti-CD20 antibody, an anti-IgM antibody, or an anti-IgD antibody. In some aspects, the collected cells comprise lymphocytes. In some aspects, the lymphocytes are B-cells.

The disclosure provides a method for identifying a subject at risk for the development of lung cancer comprising: (a) obtaining a test sample from a human subject,; (b) performing a circulating tumor cell (CTC) enrichment step comprising: (i) removing plasma from the sample, (ii) removing erythrocytes from the sample, (iii) contacting the sample with at least one biotinylated affinity agent that binds a cell surface marker, and (iv) contacting the sample with streptavidin-coated magnetic particles and collecting cells from the sample that express the cell surface marker; (c) hybridizing the enriched cells in the sample with labeled nucleic acid probes that hybridize to regions of chromosomal DNA; (d) evaluating the signal pattern for the selected cells by detecting fluorescence in situ hybridization from cells; (e) detecting CTCs based on the pattern of hybridization to the labeled nucleic acid probes to said selected cells; and (f) identifying the subject at risk for the development of lung cancer when the number of CTC per sample is above a predetermined cutoff value.

In some aspects, the cell surface marker is a B-cell specific cell surface marker. In some aspects, the B-cell specific cell surface marker is CD19. In some aspects, the at least one biotinylated affinity agent comprises an anti-CD19 antibody.

In some aspects, positive and negative selection methods can be combined. For example, cells expressing one or more cell surface markers can be depleted from the sample (negative selection) followed by collection (positive selection) of cells expressing one or more additional surface markers.

### Cell Cryopreservation and Ampule Thawing

In some aspects, blood cells including leukocytes not used in the CTC enrichment procedure are fixed with a paraformaldehyde solution and washed once with PBS containing 10% FBS. In aspects, the cells are resuspended in 1 mL cryopreservation medium containing 10% DMSO and slowly frozen in a -80° C. freezer (-1° C./min) and then transferred to liquid nitrogen. In some aspects, aliquots of frozen cells are thawed in a 37° C. water bath for approximately 2 minutes, followed by two washes with 10 mL PBS containing 10% FBS to reduce DMSO.

### Fluorescence In-Situ Hybridization

In some aspects, the methods of the disclosure further comprise contacting the cells following CTC enrichment with a labelled nucleic acid probe, and detecting hybridized cells by fluorescence in situ hybridization. In some aspects, the nucleic acid probes are specific for any genetic marker that is most frequently amplified or deleted in CTCs. In some aspects, the nucleic acid probes are specific to 3p22.1, 10q22.3, chromosome 10 centromeric (cep10), 3q29 or chromosome 3 centromeric (cep3). In some aspects, the labeled nucleic acid probes for 3p22.1 is an RPL14, CD39L3, PMGM, or GC20 probe. In some aspects, the labeled nucleic acid probes for 10q22.3 is a surfactant protein A1 or surfactant protein A2 probe.

In some aspects, following CTC enrichment, the cells are fixed with Carnoy’s fixative (3:1 solution of methanol and glacial acetic acid) for 30 minutes. In some aspects, the cells are fixed using 95% ethanol. Following cell fixation the sample is contacted with a protease. In some aspects, the protease is pepsin. Following incubation with a protease, the sample is contacted with labelled nucleic acids.

### CTC Identification

In some aspects, a CTC is identified when the hybridization pattern of the nucleic acid probes depicts a gain of two or more chromosomal regions in a cell. In some aspects, a CTC is identified when the hybridization pattern of the nucleic acid probes depicts a loss of two or more chromosomal regions in a cell.

In some aspects, a cell is classified as normal if the FISH hybridization pattern shows 2 spots of each color indicating two copies of each nucleic acid probe. In some aspects, a deletion is a loss of one or more spots belonging to a nucleic acid probe indicating a deletion of a target genetic sequence. In some aspects, a gain is the appearance of an additional spot belonging to a nucleic acid probe indicating a duplication of a target genetic sequence. In some aspects, a CTC is defined as a gain of two or more different nucleic acid probes.

### Image Acquisition and Analysis

In some aspects, Slides containing cells are imaged using a Bioview Allegro-Plus microscope system (Bioview USA, Billerica, MA). In some aspects, images are acquired using a 60x objective (Olympus, UPlanSapo, 1.35 NA oil immersion) and a FLIR Grasshopper 3 monochrome camera (12-bit, 2448 × 2048 pixels, 3.4 µm pixel size) controlled using Bioview Duet software. In some aspects, all cells are imaged with 21 transverse sections spanning 0.65 µm.

In some aspects, objects were classified by the Bioview Duet software according to probe copy number variation (“normal” cells show 2 spots of each color, “deletion” is a loss of one or more spots, “single-gain” is an extra spot in one color, and “CTC” is defined as a gain in two or more channels). In some aspects, a licensed technician analyzes cells binned in the “CTC” class by the Bioview Duet software to verify each cell. CTC counts are normalized by dividing the CTC count by the total number of cells analyzed and multiplying by 10,000. A minimum of 10,000 cells are analyzed per subject. Total CTC count, total cell count, and normalized CTC counts were sent for unblinding for each subject

### Cancer Risk Assessment

In some aspects, a CTC count greater than 0.5 CTC/10,000 cells represents a risk of lung cancer. In some aspects, a CTC count greater than 1 CTC/10,000 cells represents a risk of lung cancer. In some aspects, a CTC count greater than 2 CTC/10,000 cells represents a risk of lung cancer. In some aspects, a CTC count greater than 3 CTC/10,000 cells represents a risk of lung cancer. In some aspects, a CTC count greater than 4 CTC/10,000 cells represents a risk of lung cancer. In some aspects, a CTC count greater than 5 CTC/10,000 cells represents a risk of lung cancer. In some aspects, a CTC count greater than 10 CTC/10,000 cells represents a risk of lung cancer. In some aspects, a CTC count greater than 20 CTC/10,000 cells represents a risk of lung cancer.

In some aspects, the subject with a CTC count greater than 5 CTC/10,000 cells is referred for surgical resection of the nodule.

The disclosure provides methods of evaluating cancer in a subject comprising determining the level of circulating tumor cells (CTCs) in a sample containing blood cells from the patient by methods of the disclosure, wherein a higher level of CTCs in the sample, as compared to a control or predetermined number of CTCs from a non-aggressive form of cancer, is indicative of an aggressive form of cancer and/or a poor cancer prognosis.

The disclosure provides methods of staging cancer in a subject comprising determining circulating tumor cells (CTC) in a sample containing blood cells from the subject by methods of the disclosure, wherein a higher level of CTCs in the sample as compared to a predetermined control for a given stage is indicative of a more advanced stage of cancer, and a lower level of CTCs in the sample as compared to a control for a given stage is indicative of a less advanced stage of cancer.

### Cancer

The present disclosure envisions the use of assays to detect cancer and predict its progression in conjunction with cancer therapies. In some cases, where patients are suspected to be at risk of cancer, prophylactic treatments may be employed. In other cancer subjects, diagnosis may permit early therapeutic intervention. In yet other situations, the result of the assays described herein may provide useful information regarding the need for repeated treatments, for example, where there is a likelihood of metastatic, recurrent or residual disease. Finally, the present disclosure may prove useful in demonstrating which therapies do and do not provide benefit to a particular patient.

Furthermore, the methods described in this application are able to be translated into a method for isolating circulating tumor cells from any other type of cancer that gives rise to blood borne metastases. This would include cancers of lung, breast, colon, prostate, pancreas, esophagus, all gastro-intestinal tumors, urogenital tumors, kidney cancers, melanomas, endocrine tumors, sarcomas, etc.

The current invention is useful for the prognosis and diagnosis of lung cancers, which can be defined by a number of histologic classifications including: squamous cell carcinomas such as squamous carcinoma; small cell carcinomas such as oat cell carcinoma, intermediate cell type carcinoma, combined oat and cell carcinoma; adenocarcinomas such as acinar adenocarcinoma, papillary adenocarcinoma, bronchioloalveolar carcinoma, and solid carcinoma with mucus formation; large cell carcinoma such as giant cell carcinoma and clear cell carcinoma; adenosquamous carcinoma; carcinoid; and bronchial gland carcinomas such as adenoid cystic, and mucoepidermoid carcinoma. Diagnosis and prognosis of other smoking related cancers is possible with these probes. Squamous cell carcinoma of the head and neck has the same risk factors as lung cancer and is hypothesized to have similar etiology (Shriver, 1998). Similarly, smoking is an etiological factor for cancer of the bladder, head, neck, kidneys, pancreas, and cancer of the upper airways including cancer of the mouth, throat, pharynx, larynx, or esophagus.

### A. Tumorigenesis

The deletion of various genes in tumor tissue has been well studied in the art. However, there remains a need for probes that are significant for detecting early molecular events in the development of cancers, as well as molecular events that make patients susceptible to the development of cancer. Probes used for the staging of cancer are also of interest. The proposed sequence leading to tumorigenesis includes genetic instability at the cellular or submicroscopic level as demonstrated by loss or gain of chromosomes, leading to a hyperproliferative state due to theoretical acquisition of factors that confer a selective proliferative advantage. Further, at the genetic level, loss of function of cell cycle inhibitors and tumor suppressor genes (TSG), or amplification of oncogenes that drive cell proliferation, are implicated.

Following hyperplasia, a sequence of progressive degrees of dysplasia, carcinoma-in-situ and ultimately tumor invasion is recognized on histology. These histologic changes are both preceded and paralleled by a progressive accumulation of genetic damage. At the chromosomal level genetic instability is manifested by a loss or gain of chromosomes, as well as structural chromosomal changes such as translocation and inversions of chromosomes with evolution of marker chromosomes. In addition cells may undergo polyploidization. Single or multiple clones of neoplastic cells may evolve characterized in many cases by aneuploid cell populations. These can be quantitated by measuring the DNA content or ploidy relative to normal cells of the patient by techniques such as flow cytometry or image analysis.

### B. Prognostic Factors and Staging

The stage of a cancer at diagnosis is an indication of how much the cancer is spread and can be one of the most important prognostic factors regarding patient survival. Staging systems are specific for each type of cancer. For example, at present the most important prognostic factor regarding the survival of patients with lung cancer of non-small cell type is the stage of disease at diagnosis. For example, the most important prognostic factor regarding the survival of patients with lung cancer of non-small cell type is the stage of disease at diagnosis. Conversely, small cell cancer usually presents with wide spread dissemination hence the staging system is less applicable. The staging system was devised based on the anatomic extent of cancer and is now known as the TNM (Tumor, Node, Metastasis) system based on anatomical size and spread within the lung and adjacent structures, regional lymph nodes and distant metastases. The only hope presently for a curative procedure lies in the operability of the tumor which can only be resected when the disease is at a low stage when confined to the organ of origination.

### C. Grading of Tumors

The histological type and grade of lung cancers do have some prognostic impact within the stage of disease with the best prognosis being reported for stage I adenocarcinoma, with 5 year survival at 50% and 1-year survival at 65% and 59% for the bronchiolar-alveolar and papillary subtypes (Naruke et al., 1988; Travis et al., 1995; Carriaga et al., 1995). For squamous cell carcinoma and large cell carcinoma the 5 year survival is around 35%. Small cell cancer has the worst prognosis with a 5 year survival rate of only 12% for patients with localized disease (Carcy et al., 1980; Hirsh, 1983; Vallmer et al., 1985). For patients with distant metastases survival at 5 years is only 1-2% regardless of histological subtype (Naruke et al., 1988). In addition to histological subtype, it has been shown that histological grading of carcinomas within subtype is of prognostic value with well differentiated tumors having a longer overall survival than poorly differentiated neoplasms. Well differentiated localized adenocarcinoma has a 69% overall survival compared to a survival rate of only 34% of patients with poorly differentiated adenocarcinoma (Hirsh, 1983). The 5 year survival rates of patients with localized squamous carcinoma have varied from 37% for well differentiated neoplasms to 25% for poorly differentiated squamous carcinomas (Ihde, 1991).

The histologic criteria for subtyping lung tumors are as follows: squamous cell carcinoma consists of a tumor with keratin formation, keratin pearl formation, and/or intercellular bridges. Adenocarcinomas consist of a tumor with definitive gland formation or mucin production in a solid tumor. Small cell carcinoma consists of a tumor composed of small cells with oval or fusiform nuclei, stippled chromatin, and indistinct nuclei. Large cell undifferentiated carcinoma consists of a tumor composed of large cells with vesicular nuclei and prominent nucleoli with no evidence of squamous or glandular differentiation. Poorly differentiated carcinoma includes tumors containing areas of both squamous and glandular differentiation.

### D. Development of Carcinomas

The evolution of carcinoma of the lung is most likely representative of a field cancerization effect as a result of the entire aero-digestive system being subjected to a prolonged period of carcinogenic insults such as benzylpyrenes, asbestosis, air pollution and chemicals other carcinogenic substances in cigarette smoke or other environmental carcinogens. This concept was first proposed by Slaughter et al. (1953). Evidence for existence of a field effect is the common occurrence of multiple synchronous for metachronous second primary tumors (SPTs) that may develop throughout the aero-digestive tract in the oropharynx, upper esophagus or ipsilateral or contralateral lung.

Accompanying these molecular defects is the frequent manifestation of histologically abnormal epithelial changes including hyperplasia, metaplasia, dysplasia, and carcinoma-in-situ. It has been demonstrated in smokers that both the adjacent normal bronchial epithelium as well as the preneoplastic histological lesions may contain clones of genetically altered cells (Wistuba et al., 2000).

Licciardello et al. (1989) found a 10-40% incidence of metachronous tumors and a 9-14% incidence of synchronous SPTs in the upper and lower aero-digestive tract, mostly in patients with the earliest primary tumors SPTs may impose a higher risk than relapse from the original primary tumor and may prove to be the major threat to long term survival following successful therapy for early stage primary head, neck or lung tumors. Hence it is vitally important to follow these patients carefully for evidence of new SPTs in at risk sites for new malignancies specifically in the aero-digestive system.

In addition to chromosomal changes at the microscopic level, multiple blind bronchial biopsies may demonstrate various degrees of intraepithelial neoplasia at loci adjacent to the areas of lung cancer. Other investigators have shown that there are epithelial changes ranging from loss of cilia and basal cell hyperplasia to CIS in most light and heavy smokers and all lungs that have been surgically resected for cancer (Auerbach et al., 1961). Voravud et al. (1993) demonstrated by in-situ hybridization (ISH) studies using chromosome-specific probes for chromosomes 7 and 17 that 30-40% of histologically normal epithelium adjacent to tumor showed polysomies for these chromosomes. In addition there was a progressive increase in frequency of polysomies in the tissue closest to the carcinoma as compared to normal control oral epithelium from patients without evidence of carcinoma. The findings of genotypic abnormalities that increased closer to the area of the tumor support the concept of field cancerization. Interestingly, there was no increase in DNA content as measured in the normal appearing mucosa in a Feulgen stained section adjacent to the one where the chromosomes were measured, reflecting perhaps that insufficient DNA had been gained in order to alter the DNA index. Interestingly, a very similar increase in DNA content was noted both in dysplastic areas close to the cancer and in the cancerous areas suggesting that complex karyotypic abnormalities that are clonal have already been established in dysplastic epithelium adjacent to lung cancer. Others have also shown an increase in the number of cells showing p53 mutations in dysplastic lesions closest to areas of cancer, which are invariably also p53 mutated. Other chromosomal abnormalities that have recently been demonstrated in tumors and dysplastic epithelium of smokers includes deletions of 3p, 17p, 9 p and 5q (Feder et al., 1998; Yanagisawa et al., 1996; Thiberville et al., 1995).

### E. Chromosome Deletions in Lung Cancer

Small cell lung cancer (SCLC) and non-small cell lung cancer commonly display cytogenetically visible deletions on the short arm of chromosome 3 (Hirano et al., 1994; Valdivieso et al., 1994; Cheon et 41993; Pence et al., 1993). This 3p deletion occurs more frequently in the lung tumor tissues of patients who smoke than it does in those of nonsmoking patients. (Rice et al., 1993) Since approximately 85% lung cancer patients were heavy cigarette smokers (Mrkve et al., 1993), 3p might contain specific DNA loci related to the exposure of tobacco carcinogens. It also has been reported that 3p deletion occurs in the early stages of lung carcinogenesis, such as bronchial dysplasia (Pantel et al., 1993). In addition to cytogenetic visible deletions, loss of heterozygosity (LOH) studies have defined 3-21.3 as one of the distinct regions that undergo loss either singly or in combination (Fontanini et al., 1992; Liewald et al., 1992). Several other groups have found large homozygous deletions at 3p21.3 in lung cancer (Macchiarini et al., 1992; Miyamoto et al., 1991; Ichinose et al., 1991; Yamaoka et al., 1990). Transfer of DNA fragments from 3-21.3-3p21.2 into lung tumor cell lines could suppress the tumorigenesis (Sahin et al., 1990; Volm et al., 1989). These findings strongly suggest the presence of at least one tumor suppressor gene in this specific chromosome region whose loss will initiate lung carcinogenesis.

Cytogenetic observation of lung cancer has shown an unusual consistency in the deletion rate of chromosome 3p. In fact, small cell lung cancer (SCLC) demonstrates a 100% deletion rate within certain regions of chromosome 3p. Non small cell lung cancer (NSCLC) demonstrates a 70% deletion rate (Mitsudomi et al., 1996; Shiseki et al., 1996). Loss of heterozygosity and comparative genomic hybridization analysis have shown deletions between 3p14.2 and 3p21.3 to be the most common finding for lung carcinoma and is postulated to be the most crucial change in lung tumorigenesis (Wu et al., 1998). It has been hypothesized that band 3p21.3 is the location for lung cancer tumor suppressor genes. The hypothesis is supported by chromosome 3 transfer studies, which reduced tumorigenicity in lung adenocarcinoma.

Allelotype studies on non-small cell lung carcinoma indicated loss of genetic material on chromosome 10q in 27% of cases. Studies of chromosome 10 allelic loss have shown that there is a very high incidence of LOH in small cell lung cancer, up to 91% (Alberola et al., 1995; Ayabe et al., 1994). A statistically significant LOH of alleles on 10q was noted in metastatic squamous cell carcinoma (SCC) in 56% of cases compared to non-metastatic SCC with LOH seen in only 14% of cases (Ayabe et al., 1994). No LOH was seen in other subtypes on NSCLC. Additionally, using microsatellite polymorphism analysis, it was shown that a high incidence of loss exists between D10s677 and D1051223. This region spans the long arm of chromosome 10 at bands q21-q24 and overlaps the region deleted in the a study of advanced stage high grade bladder cancers which demonstrated a high frequency of allele loss within a 2.5 cM region at 10q22.3-10q23.1 (Kim et al., 1996).

### Sorting and Selection by Nuclear Size

In one aspect, the disclosure provides for isolating and/or classifying CTCs according to nuclear size or nucleus/cytoplasm ratio. These methods may involve physical sorting, such as by FACS or other nuclei sorting means, but analysis of optical data using a computer-driven size analysis, or by manual interrogation of cell nuclei, such as by using standard light microscopy. Typically, the nuclei are stained in order to permit assessment/sorting, such as with DAPI (4′,6-diamidino-2-phenylindoie). In certain embodiments, the nuclei will be obtained from cells and sorted on their own. Cells may be lysed using standard cell lysis protocols.

### A. Bioview System and Software

The Bioview Duet™ (Rehovot, Israel) system uses a color or monochromatic CCD cameras normally images and classifies all nucleated cells presented on the cytopreparation. The number of cells classified is preset by the operator however usually several thousand cells are scanned. There is a “research” mode or an open software system, that then records for each cell:
1) nuclear area in pixels, based on the DAPI stain, expressed as arbitrary units, thus, if 5000 it means that the cell area is 5000 pixels;
2) nuclear diameter; and
3) circularity factor (CFs), calculated by modifying the elongation (proportion between the height and the width of the cell) where a perfect circle will have the value of 1 (lymphocytes have CFs close to 1, abnormal cells have much CFs >>1, due to their nuclear perimeter irregularity).

In order to increase the yield of CTCs, the inventor made the following measurements and then adjusted the software so as to enhance the yield of abnormal cells and decrease the numbers of normal lymphocytes.

The nuclear area for the abnormal (malignant CTCs) cells was based on the number of pixels occupied by the nucleus (as defined by FISH polysomy >2) as measured on the DAPI stain (a nuclear stain) and was expressed in arbitrary units.

The nuclear area for the lymphocytes was the number of pixels occupied by the lymphocytes in the blood that were diploid by FISH, with a circularity factor close to 1. The way the measurement was derived was from observing the average nuclear pixel area of the lymphocytes from numerous malignant specimens (“internal” control lymphocytes) as well as recording the average nuclear pixel area of lymphocytes within control specimens or “external” control lymphocytes, from patients known to be healthy without history of prior malignancy or malignant cells in their blood streams. Similarly, observations were recorded of the nuclear area of numerous “abnormal” cells (circulating tumor cells) defined as cells with 2 or more polysomies (extra chromosomes) from patients with known lung cancer. The inventor showed that the nuclear areas for the CTCs far exceeded the arbitrary threshold, as discussed below.

In embodiments where absolute numbers of CTCs are diagnostic, a finding of 4 or more CTCs will indicate that the patient has cancer. The inventor notes that some patients in remission for several years can show a few CTCs (minimal residual disease; less than 4 CTCs as defined herein) which may represent dormant CTCs. The half-life of CTCs is said to be about 4-8 hours, so there is a constantly replenishing source. This is currently a phenomenon of great biological relevance, as after several years of apparent “remission” patients can relapse and die, very likely implicating these dormant CTCs.

### B. Threshold

A threshold of 78 was chosen based on the average pixel area of lymphocytes with a CF close to 1, within the blood from patients who had lung cancer. This threshold value was significantly lower than the average pixels noted for abnormal cells (defined by FISH polysomy >2).

### C. Classification

A duplicate task with exclusions was created so that the system would only start classifying cells within the Ficoll purified specimen that were larger than 78. Thus, all cells less than 78, comprising the average nuclear area of lymphocytes were excluded, and only the cells that meet the derived criterion (threshold >78) were classified and presented to the operator for interactive evaluation. In addition, the Bioview system creates a pie chart to display diploid cells, aneuploid cells (single gains or losses) and abnormal cells (at least polysomy of 2 or more genes as defined by FISH probes, 3cen, 3p, 3q, 10cen and 10q).

The instrument task is set to scan several thousand cells so that at least 500 intact and non-overlapped cells with the derived criterion (threshold >78) can be selected from several thousand images, which are presented to the operator for interactive evaluation of extra signals (gains) or loss of signals (deletions).

When evaluating the scanned cells, the operator will first check different categories of cells according to the pie chart, beginning with the “abnormal” cells which are defined as at least 2 chromosomes with extra copies, then the single gain and loss categories, and finally the remaining cells will be interactively analyzed until 500 cells have been scored.

### Gene Probes

The present disclosure comprises contacting the selected cells with a labeled nucleic acid probe, and detecting hybridized cells by fluorescence in situ hybridization. These probes may be specific for any genetic marker that is most frequently amplified or deleted in CTCs. In particular, the probes may be a 3p22.1 probe, which is a nucleic acid probe targeting RPL14, CD39L3, PMGM, or GC20, combined with centromeric 3; a 10q22-23 probe (encompassing surfactant protein A1 and A2) combined with centromeric 10; or a PI3 kinase probe. Other genetic markers may include, but are not limited to, centromeric 3, 7, 17, 9p21, 5p15.2, EGFR, C-myc8q22, and 6p22-22. For a further discussion of gene probes see U.S. Publication No. 2007/0218480, herein incorporated by reference in its entirety.

### 3P22.1 Gene Probes

A 3p22.1 probe is a nucleic acid probe targeting RPL14, CD39L3, PMGM, or GC20, combined with centromeric 3. The human ribosomal L14 (RPL14) gene (GenBank Accession NM_003973), and the genes CD39L3 (GenBank Accession AAC39884 and AF039917), PMGM (GenBank Accession P15259 and J05073), and GC20 (GenBank Accession NM_005875) were isolated from a BAC (GenBank Accession AC104186, herein incorporated by reference) and located in the 3p22.1 band within the smallest region of deletion overlap of various lung tumors. The RPL14 gene sequence contains a highly polymorphic trinucleotide (CTG) repeat array, which encodes a variable length polyalanine tract. Polyalanine tracts are found in gene products of developmental significance that bind DNA or regulate transcription. For example, Drosophila proteins Engraled, Kruppel and Even-Skipped all contain polyalanine tracts that act as transcriptional repressors. It is understood that the polyalanine tract plays a key role in the nonsense-mediated mRNA decay pathway that rids cells aberrant proteins and transcripts. Genotype analysis of RPL14 shows that this locus is 68% heterozygous in the normal population, compared with 25% in NSCLC cell lines. Cell cultures derived from normal bronchial epithelium show a 65% level of heterozygosity, reflecting that of the normal population. See also RP11-391M1/AC104186.

Genes with a regulatory function such as the RPL14 gene, along with the genes CD39L3, PMGM, and GC20 and analogs thereof, are good candidates for diagnosis of tumorigenic events. It has been postulated that functional changes of the RPL14 protein can occur via a DNA deletion mechanism of the trinucleotide repeat encoding for the protein. This deletion mechanism makes the RPL14 gene an attractive sequence that may be used as a marker for the study of lung cancer risk (Shriver et al., 1998). In addition, the RPL14 gene shows significant differences in allele frequency distribution in ethnically defined populations, making this sequence a useful marker for the study of ethnicity adjusting lung cancer (Shriver et al., 1998). Therefore, this gene is useful in the early detection of lung cancer, and in chemopreventive studies as an intermediate biomarker.

### 3P21.3 Gene Probes

### Structural Features

Recently, the human ribosomal L14 (RPL14) gene (GenBank Accession NM_003973, SEQ ID NO: 1), and the genes CD39L3 (GenBank Accession AAC39884 and AF039917; SEQ ID NO: 3), PMGM (GenBank Accession P15259 and J05073; SEQ ID NO: 5), and GC20 (GenBank Accession NM—005875; SEQ ID NO: 7) were isolated from a BAC (GenBank Accession AC019204, herein incorporated by reference) and located in the 3p21.3 band within the smallest region of deletion overlap of various lung tumors. The RPL14 gene sequence contains a highly polymorphic trinucleotide (CTG) repeat array, which encodes a variable length polyalanine tract. Polyalanine tracts are found in gene products of developmental significance that bind DNA or regulate transcription. For example, Drosophila proteins Engraled, Kruppel and Even-Skipped all contain polyalanine tracts that act as transcriptional repressors. Genotype analysis of RPL14 shows that this locus is 68% heterozygous in the normal population, compared with 25% in NSCLC cell lines. Cell cultures derived from normal bronchial epithelium show a 65% level of heterozygosity, reflecting that of the normal population. Functional Aspects

Genes with a regulatory function such as the RPL14 gene (SEQ ID NO: 1), along with the genes CD39L3, PMGM, and GC20 (SEQ ID NOS: 3, 5 and 7) and analogs thereof, are good candidates for diagnosis of tumorigenic events. It has been postulated that functional changes of the RPL14 protein (SEQ ID NO: 2) can occur via a DNA deletion mechanism of the trinucleotide repeat encoding for the protein. This deletion mechanism makes the RPL14 gene an attractive sequence that may be used as a marker for the study of lung cancer risk (Shriver et al., 1998). In addition, the RPL14 gene shows significant differences in allele frequency distribution in ethnically defined populations, making this sequence a useful marker for the study of ethnicity adjusting lung cancer (Shriver et al., 1998). Therefore, this gene is useful in the early detection of lung cancer, and in chemopreventive studies as an intermediate biomarker.

### 10q22 Gene Probes

### Structural Features

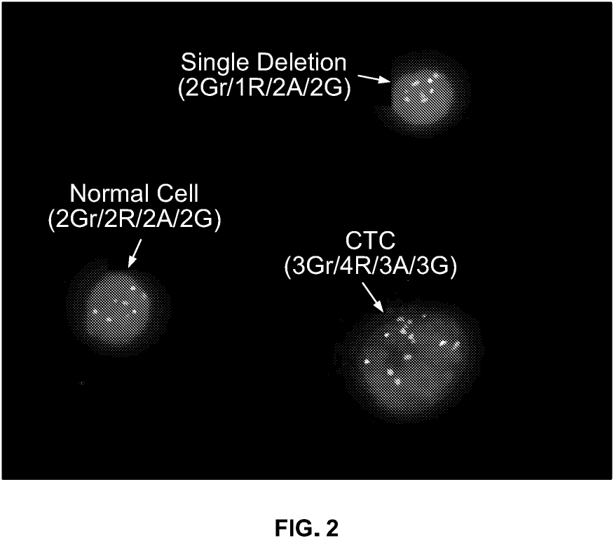

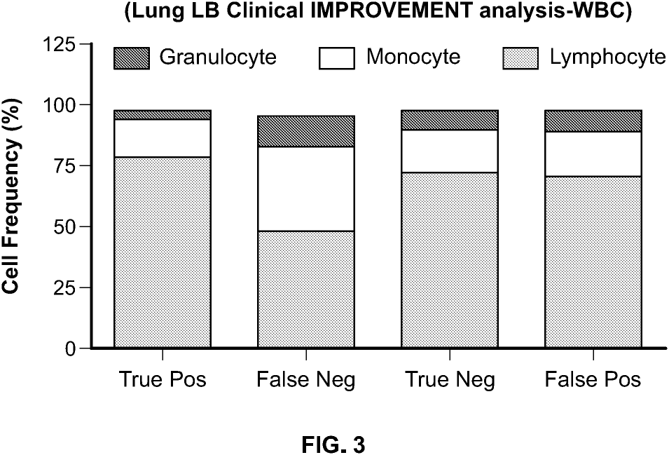

In other embodiments, the probe may be a 10q22-23 probe, which encompasses surfactant protein A1 and A2, combined with centromeric 10. The 10q22 BAC (46b12) is 200 Kb and is adjacent and centromeric to PTEN/MMAC1 (GenBank Accession AF067844), which is at 10q22-23 and can be purchased through Research Genetics (Huntsville, Ala.) (FIG. 3). Alterations to 10q22-25 has been associated with multiple tumors, including lung, prostate, renal, and endometrial carcinomas, melanoma, and meningiomas, suggesting the possible suppressive locus affecting several cancers in this region. The PTEN/MMAC1 gene, encoding a dual-specificity phosphatase, is located in this region, and has been isolated as a tumor suppressor gene that is altered in several types of human tumors including brain, bladder, breast and prostate cancers. PTEN/MMAC1 mutations have been found in some cancer cell lines, xenografts, and hormone refractory cancer tissue specimens. Because the inventor’s 10q22 BAC DNA sequence is adjacent to this region, the DNA sequences in the BAC 10q22 may be involved in the genesis and/or progression of human lung cancer. See also RP11-506M13/AC068139.6

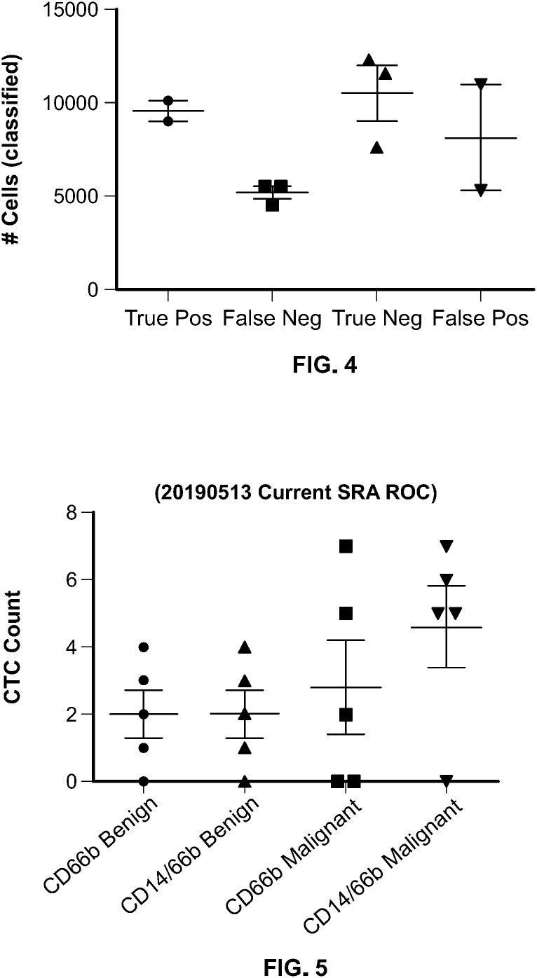

Pulmonary-associated surfactant protein A1 (SP-A) is located at 10q22.3. Surfactant protein-A-phospholipid-protein complex lowers the surface tension in the alveoli of the lung and plays a major role in host defense in the lung. Surfactant protein-A1 is also present in alveolar type-2 cells, which are believed to be putative stem cells of the lung. It is known that type-2 cells participate in repair and regeneration after alveolar damage. Thus, it is possible that the type-2 cells express telomerase and C-MYC, which leads to the loss of the surfactant protein and the development of non-small cell lung cancer (FIG. 4). The 10q22 probe is useful in the further development of clinical biomarkers for the early detection of neoplastic events, for risk assessment and monitoring the efficacy of chemoprevention therapy.

### Functional Aspects

Functional evidence for the presence of tumor suppressor genes on 10q has been provided by microcell-mediated chromosomal transfer. The resulting hybrid clones displayed a suppressed tumorigenic phenotype with the inability to proliferate in nude mice and soft agarose. Sequence analysis of the PTEN/MMAC1 gene in lung cancer revealed a G to C substitution located 8 bp upstream of the coding region of exon1 and which seems to be a polymorphism, in 4 of the 30 cases of lung cancer tested. Somatic mutations of the TPEN/MMAC1 gene were not identified in any of the tumors at the primary and metastatic sites of lung cancer, indicating that point mutations in the PTEN/MMAC1 gene are probably not an important factor in tumorigenesis and the progression of a major subset of lung cancers. Other more important tumor suppressor genes must lie close to the PTEN/MMAC1 gene, in the vicinity of the inventors’ 10q22 BAC locus. Therefore, the 10q22 probe is useful in the further development of clinical biomarkers for the early detection of neoplastic events, for risk assessment and monitoring the efficacy of chemoprevention therapy in high risk former or current smokers.

### C. Commercial Probe Sets

Any commercial probes or probe sets may also be used with the present disclosure. For example, the UroVysion DNA probe set (Vysis/Abbott Molecular, Des Plaines, Ill.) may be used, which includes probes directed to centromeric 3, centromeric 7, centromeric 17, 9p21.3. It has been established that UroVysion probes detect early changes of lung cancer. In other embodiments, the LaVysion DNA probe set (Vysis/Abbott Molecular, Des Plaines, Ill.), which includes probes to 7p12 (epidermal growth factor receptor); 8q24.12-q24.13 (MYC); 6p11.1-q11 (chromosome enumeration (Probe CEP 6); and Sp15.2 (encompassing the SEMA5A gene), may be used. It has been noted that the LaVysion probe set detects higher stages or more advanced stages of lung cancer. Furthermore, a single probe set directed to centromeric 7/7p12 (epidermal growth factor receptor) may also be used with the present disclosure.

### Methods for Assessing Gene Structure

In accordance with the present disclosure, one will utilize various probes to examine the structure of genomic DNA from patient samples. A wide variety of methods may be employed to detect changes in the structure of various chromosomal regions. The following is a non-limiting discussion of such methods.

### A. Fluorescence In Situ Hybridization and Chromogenic In Situ Hybridization

Fluorescence in situ hybridization (FISH) can be used for molecular studies. FISH is used to detect highly specific DNA probes which have been hybridized to chromosomes using fluorescence microscopy. The DNA probe is labeled with fluorescent or non fluorescent molecules which are then detected by fluorescent antibodies. The probes bind to a specific region or regions on the target chromosome. The chromosomes are then stained using a contrasting color, and the cells are viewed using a fluorescence microscope.

Each FISH probe is specific to one region of a chromosome, and is labeled with fluorescent molecules throughout its length. Each microscope slide contains many metaphases. Each metaphase consists of the complete set of chromosomes, one small segment of which each probe will seek out and bind itself to. The metaphase spread is useful to visualize specific chromosomes and the exact region to which the probe binds. The first step is to break apart (denature) the double strands of DNA in both the probe DNA and the chromosome DNA so they can bind to each other. This is done by heating the DNA in a solution of formamide at a high temperature (70-75° C.). Next, the probe is placed on the slide and the slide is placed in a 37° C. incubator overnight for the probe to hybridize with the target chromosome. Overnight, the probe DNA seeks out its target sequence on the specific chromosome and binds to it. The strands then slowly reanneal. The slide is washed in a salt/detergent solution to remove any of the probe that did not bind to chromosomes and differently colored fluorescent dye is added to the slide to stain all of the chromosomes so that they may then be viewed using a fluorescent light microscope. Two, or more different probes labeled with different fluorescent tags can be mixed and used at the same time. The chromosomes are then stained with a third color for contrast. This gives a metaphase or interphase cell with three or more colors which can be used to detect different chromosomes at the same time, or to provide a control probe in case one of the other target sequences are deleted and a probe cannot bind to the chromosome. This technique allows, for example, the localization of genes and also the direct morphological detection of genetic defects.

The advantage of using FISH probes over microsatellite instability to test for loss of allelic heterozygosity is that the:
(a) FISH is easily and rapidly performed on cells of interest and can be used on paraffin-embedded, or fresh or frozen tissue allowing the use of micro-dissection;
(b) specific gene changes can be analyzed on a cell by cell basis in relationship to centromeric probes so that true homozygosity versus heterozygosity of a DNA sequence can be evaluated (use of PCR™ for microsatellite instability may permit amplification of surrounding normal DNA sequences from contamination by normal cells in a homozygously deleted region imparting a false positive impression that the allele of interest is not deleted);
(c) PCR cannot identify amplification of genes; and
(d) FISH using bacterial artificial chromosomes (BACs) permits easy detection and localization on specific chromosomes of genes of interest which have been isolated using specific primer pairs.

Chromogenic in situ hybridization (CISH) enables the gain of genetic information in the context of tissue morphology using methods already present in histology labs. CISH allows detection of gene amplification, chromosome translocations and chromosome number using conventional enzymatic reactions under the brightfield microscope on formalin-fixed, paraffin-embedded (FFPE) tissues. U.S. Publication No. 2009/0137412, incorporated herein by reference. The scanning may be performed, for example, on an automated scanner with Fluorescence capabilities (Bioview System, Rehovot, Israel).

### B. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present disclosure. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present disclosure (Walker et al., 1992).

Strand Displacement Amplification (SDA) is another method of carrying out isothermal amplification of nucleic acids, which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present disclosure. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA’s are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present disclosure. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present disclosure (Wu et al., 1989, incorporated herein by reference in its entirety).

### C. Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above.

### D. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present disclosure: adsorption, partition, ion-exchange and molecular sieve, and many specialized techniques for using them including column, paper, thin-layer and gas chromatography (Freifelder, 1982).

### E. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present disclosure.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994). The present disclosure provides methods by which any or all of these types of analyses may be used.

### F. Kit Components

All the essential materials and reagents required for detecting changes in the chromosomal regions discussed above may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification, and optionally labeling agents such as those used in FISH. Such kits also generally will comprise, in suitable means, distinct containers for each individual reagent and enzyme as well as for each primer or probe.

### G. Chip Technologies

Specifically contemplated by the present inventors are chip-based DNA technologies such as those described by Hacia et al. (1996) and Shoemaker et al. (1996). These techniques involve quantitative methods for analyzing large numbers of genes rapidly and accurately. By tagging genes with oligonucleotides or using fixed probe arrays, one can employ chip technology to segregate target molecules as high density arrays and screen these molecules using methods such as fluorescence, conductance, mass spectrometry, radiolabeling, optical scanning, or electrophoresis. See also Pease et al. (1994); Fodor et al. (1991).

Biologically active DNA probes may be directly or indirectly immobilized onto a surface to ensure optimal contact and maximum detection. When immobilized onto a substrate, the gene probes are stabilized and therefore may be used repetitively. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules (Saiki et al., 1994).

Immobilization of the gene probes may be achieved by a variety of methods involving either non-covalent or covalent interactions between the immobilized DNA comprising an anchorable moiety and an anchor. DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. Gene probe may be bound directly to membranes using ultraviolet radiation. With nitrocellous membranes, the probes are spotted onto the membranes. A UV light source is used to irradiate the spots and induce cross-linking. An alternative method for cross-linking involves baking the spotted membranes at 80° C. for two hours in vacuum.

Immobilization can consist of the non-covalent coating of a solid phase with streptavidin or avidin and the subsequent immobilization of a biotinylated polynucleotide (Holmstrom, 1993). Precoating a polystyrene or glass solid phase with poly-L-Lys or poly L-Lys, Phe, followed by the covalent attachment of either amino- or sulfhydryl-modified polynucleotides using bifunctional crosslinking reagents (Running, 1990; Newton, 1993) can also be used to immobilize the probe onto a surface.

Immobilization may also take place by the direct covalent attachment of short, 5′-phosphorylated primers to chemically modified polystyrene plates (“Covalink” plates, Nunc) Rasmussen, (1991). The covalent bond between the modified oligonucleotide and the solid phase surface is introduced by condensation with a water-soluble carbodiimide. This method facilitates a predominantly 5′-attachment of the oligonucleotides via their 5′-phosphates.

Nikiforov et al. (U.S. Pat. No. 5,610,287) describes a method of non-covalently immobilizing nucleic acid molecules in the presence of a salt or cationic detergent on a hydrophilic polystyrene solid support containing an —OH, —C═O or —COOH hydrophilic group or on a glass solid support. The support is contacted with a solution having a pH of about 6 to about 8 containing the synthetic nucleic acid and the cationic detergent or salt. The support containing the immobilized nucleic acid may be washed with an aqueous solution containing a non-ionic detergent without removing the attached molecules.

There are two common variants of chip-based DNA technologies involving DNA microarrays with known sequence identity. For one, a probe cDNA (500^{∼}5,000 bases long) is immobilized to a solid surface such as glass using robot spotting and exposed to a set of targets either separately or in a mixture. This method, “traditionally” called DNA microarray, is widely considered as developed at Stanford University. A recent article by Ekins and Chu (1999) provides some relevant details. The other variant includes an array of oligonucleotide (20^{~}25-mer oligos) or peptide nucleic acid (PNA) probes synthesized either in situ (on-chip) or by conventional synthesis followed by on-chip immobilization. The array is exposed to labeled sample DNA, hybridized, and the identity/abundance of complementary sequences is determined. This method, “historically” called DNA chips, was developed at Affymetrix, Inc., which sells its products under the GeneChip® trademark.

### Nucleic Acids

The inventors provide a method comprising a step of contacting the selected cells with a labeled nucleic acid probe forming hybridized cells, wherein hybridization of the labeled nucleic acid is indicative of a CTC. However, the present disclosure is not limited to the use of the specific nucleic acid segments disclosed herein. Rather, a variety of alternative probes that target the same regions/polymorphisms may be employed.

### Probes and Primers

Naturally, the present disclosure encompasses DNA segments that are complementary, or essentially complementary, to target sequences. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a target nucleic acid segment under relatively stringent conditions such as those described herein. These probes may span hundreds or thousands of base pairs.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of about 8, 9, 10, 11, 12, 13, 14, 15, 16, 17, 18, 19, 20, 25, 30, 35, 40, 45, 50, 55, 60, 65, 70, 75, 80, 85, 90, 95, 100, 250, 500, 700, 722, 900, 992, 1000, 1500, 2000, 2500, 2800, 3000, 3500, 3800, 4000, 5000 or more base pairs will be used, although others are contemplated. As mentioned above, longer polynucleotides encoding 10,000, 50,000, 100,000, 150,000, 200,000, 250,000, 300,000 and 500,000 bases are contemplated. Such oligonucleotides and polynucleotides will find use, for example, as probes in FISH, Southern and Northern blots and as primers in amplification reactions.

It will be understood that this disclosure is not limited to the particular probes disclosed herein and particularly is intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present disclosure are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

### Labeling of Probes

In certain embodiments, it will be advantageous to employ nucleic acid sequences of the present disclosure in combination with an appropriate means, such as a label, for determining hybridization. A wide variety of appropriate indicator means are known in the art, including fluorescent, radioactive, chemiluminescent, electroluminescent, enzymatic tag or other ligands, such as avidin/biotin, antibodies, affinity labels, etc., which are capable of being detected. In preferred embodiments, one may desire to employ a fluorescent label such as digoxigenin, spectrum orange, fluorescein, eosin, an acridine dye, a rhodamine, Alexa 350, Alexa 430, AMCA, BODIPY 630/650, BODIPY 650/665, BODIPY-FL, BODIPY-R6G, BODIPY-TMR, BODIPY-TRX, cascade blue, Cy2, Cy3, Cy5,6-FAM, HEX, 6-JOE, Oregon green 488, Oregon green 500, Oregon green 514, pacific blue, REG, ROX, TAMRA, TET, or Texas red.

In the case of enzyme tags such as urease alkaline phosphatase or peroxidase, colorimetric indicator substrates are known which can be employed to provide a detection means visible to the human eye or spectrophotometrically, to identify specific hybridization with complementary nucleic acid-containing samples. Examples of affinity labels include but are not limited to the following: an antibody, an antibody fragment, a receptor protein, a hormone, biotin, DNP, or any polypeptide/protein molecule that binds to an affinity label and may be used for separation of the amplified gene.

The indicator means may be attached directly to the probe, or it may be attached through antigen bonding. In preferred embodiments, digoxigenin is attached to the probe before denaturation and a fluorophore labeled anti-digoxigenin FAB fragment is added after hybridization.

### Hybridization Conditions

Suitable hybridization conditions will be well known to those of skill in the art. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl2, 10 mM dithiothreitol, at temperatures between approximately 20° C. to about 37° C. Other hybridization conditions utilized could include approximately 10 mM Tris-HCl (pH 8.3), 50 mM KCl, 1.5 µM MgCl2, at temperatures ranging from approximately 40° C. to about 72° C. Formamide and SDS also may be used to alter the hybridization conditions.

### Biomarkers and Other Risk Factors

Various biomarkers of prognostic significance can be used in conjunction with the specific nucleic acid probes discussed above. These biomarkers could aid in predicting the survival in low stage cancers and the progression from preneoplastic lesions to invasive lung cancer. These markers can include proliferation activity as measured by Ki-67 (MIB 1), angiogenesis as quantitated by expression of VEGF and microvessels using CD34, oncogene expression as measured by erb B2, and loss of tumor suppresser genes as measured by p53 expression.

Multiple biomarker candidates have been implicated in the evolution of neoplastic lung lesions. Bio-markers that have been studies include general genomic markers including chromosomal alterations, specific genomic markers such as alterations in proto-oncogenes such as K-Ras, Erbβ1/EGFR, Cyclin D; proliferation markers such as Ki67 or PCNA, squamous differentiation markers, and nuclear retinoid receptors (Papadimitrakopoulou et al., 1996) The latter are particularly interesting as they may be modulated by specific chemopreventive drugs such as 13-cis-retinoic acid or 4HPR and culminate in apoptosis of the defective cells with restoration of a normally differentiated mucosa (Zou et al., 1998).

### Tumor Angiogenesis by Microvessel Counts

Tumor angiogenesis can be quantitated by microvessel density and is a viable prognostic factor in stage 1 NSCLC. Tumor microvessel density appears to be a good predictor of survival in stage 1 NSCLC.

### Vascular Endothelial Growth Factor (VEGF)

VEGF (3,6-8 ch 4) an endothelial cell specific mitogen is an important regulator of tumor angiogenesis who’s expression correlates well with lymph node metastases and is a good indirect indicator of tumor angiogenesis. VEGF in turn is upregulated by P53 protein accumulation in NSCLC.

### p53

The role of p53 mutations in predicting progression and survival of patients with NSCLC is widely debated. Although few studies imply a negligible role, the majority of the studies provide compelling evidence regarding the role of p53 as one of the prognostic factors in NSCLC. The important role of p53 in the biology of NSCLC has been the basis for adenovirus mediated p53 gene transfer in patients with advanced NSCLC (Carcy et al., 1980). In addition p53 has also been shown to be an independent predictor of chemotherapy response in NSCLC. In a recent study (Vallmer et al., 1985), the importance of p53 accumulation in preinvasive bronchial lesions from patients with lung cancer and those who did not progress to cancer were studied. It was demonstrated that p53 accumulation in preneoplastic lesions had a higher rate of progression to invasion than did p53 negative lesions.

### c-erb-B2

Similar to p53, c-erg-B2 (Her2/neu) expression has also been shown to be a good marker of metastatic propensity and an indicator of survival in these tumors.

### Ki-67 Proliferation Marker

In addition to the above markers, tumor proliferation index as measured by the extent of labeling of tumor cells for Ki-67, a nuclear antigen expressed throughout cell cycle correlates significantly with clinical outcome in Stage 1 NSCLC (Feinstein et al., 1970). The higher the tumor proliferation index the poorer is the disease free survival labeling indices provide significant complementary, if not independent prognostic information in Stage 1 NSCLC, and helps in the identification of a subset of patients with Stage 1 NSCLC who may need more aggressive therapy.

Alterations in the 3p21.3 and 10q22 loci are known to be associated with a number of cancers. More specifically, point mutations, deletions, insertions or regulatory perturbations relating to the 3p21.3 and 10q22 loci may cause cancer or promote cancer development, cause or promoter tumor progression at a primary site, and/or cause or promote metastasis. Other phenomena at the 3p21.3 and 10q22 loci include angiogenesis and tissue invasion. Thus, the present inventors have demonstrated that deletions at 3p21.3 and 10q22 can be used not only as a diagnostic or prognostic indicator of cancer, but to predict specific events in cancer development, progression and therapy.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNase protection assay, allele-specific oligonucleotide (ASO), dot blot analysis, denaturing gradient gel electrophoresis, RFLP and PCR-SSCP

Various types of defects are to be identified. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited.

### Surfactant Protein A and B

There are four main surfactant proteins: SP-A, B, C, and D. SP-A and D are hydrophilic, while SP-B and C are hydrophobic. The proteins are very sensitive to experimental conditions (temperature, pH, concentration, substances such as calcium, and so on). Moreover, their effects tend to overlap and thus it is difficult to pinpoint the specific role of each protein.

### SP-A

SP-A was the first surfactant protein to be identified, and is also the most abundant (Ingenito et al., 1999). Its molecular mass varies from 26-38 kDa (Perez-Gil et al., 1998). The protein has a “bouquet” structure of six trimers (Haagsman and Diemel, 2001), and can be found in an open or closed form depending on the other substances present in the system. Calcium ions produce the closed-bouquet form (Palaniyar et al., 1998).

SP-A plays a role in immune defense. It is also involved in surfactant transport/adsorption (with other proteins). SP-A is necessary for the production of tubular myelin, a lipid transport structure unique to the lungs. Tubular myelin consists of square tubes of lipid lined with protein (Palaniyar et al., 2001). Mice genetically engineered to lack SP-A have normal lung structure and surfactant function, and it is possible that SP-A’s beneficial surfactant properties are only evident under situations of stress (Korfhagen et al., 1996).

### SP-B

Papillary thyroid carcinoma (PTC) is clinically heterogeneous. Apart from an association with ionizing radiation, the etiology and molecular biology of PTC is poorly understood. Using oligo-based DNA arrays to study expression profiles of eight matched pairs of normal thyroid and PTC tissues, Immunohistochemical analysis detected SFTPB in 39/52 PTCs, but not in follicular thyroid carcinoma and normal thyroid tissue. Huang et al. (2001.

### Patient Interview and Other Risk Factors

In addition to analyzing the presence or absence of polymorphisms, as discussed above, it may be desirable to evaluate additional factors in a patient. For example, a patient interview, which would include a smoking history (years smoking, pack/day, etc.) is highly relevant to the diagnosis/prognosis. Also, the presence or absence of morphologic changes in sputum cells (squamous metaplasia, dysplasia, etc.) and a genetic instability score (genetic instability=composing the sum of abnormalities from various combinations in epithelial and neutrophils in sputum and/or peripheral blood cells or bone marrow cells or stem cells isolated from blood or bone marrow) may be used.

### Obtaining and Purifying Samples

In accordance with the present disclosure, one will obtain a biological sample that contains blood cells. In some embodiments, the entity evaluating the sample for CTC levels did not directly obtain the sample from the patient. Therefore, methods of the disclosure involve obtaining the sample indirectly or directly from the patient. To achieve these methods, a doctor, medical practitioner, or their staff may obtain a biological sample for evaluation. The sample may be analyzed by the practitioner or their staff, or it may be sent to an outside or independent laboratory. The medical practitioner may be cognizant of whether the test is providing information regarding a quantitative level of CTCs.

In any of these circumstances, the medical practitioner may know the relevant information that will allow him or her to determine whether the patient can be diagnosed as having an aggressive form of cancer and/or a poor cancer prognosis based on the level of CTCs. It is contemplated that, for example, a laboratory conducts the test to determine the level of CTCs. Laboratory personnel may report back to the practitioner with the specific result of the test performed.

Typically, the sample is isolated from a biological sample taken from the individual, such as a blood sample or tissue sample using standard techniques such as disclosed in Jones (1963) which is hereby incorporated by reference. Collection of the samples may be by any suitable method, although in some aspects collection is by needle, catheter, syringe, scrapings, and so forth.

The sample may be prepared in any manner known to those of skill in the art. For example, the circulating epithelial cells from peripheral blood may be isolated from the buffy layer following Ficoll-Hypaque gradient separation, allowing for enrichment of mononuclear cells (lymphocytes and epithelial cells). Other methods known to those of skill in the art may also be used to prepare the sample.

Nucleic acids may be isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. Depending on the format, the specific nucleic acid of interest is identified in the sample directly using amplification or with a second, known nucleic acid following amplification.

Following detection, one may compare the results seen in a given sample with a statistically significant reference group of samples from normal patients and patients that have or lack alterations in the various chromosome loci and control regions. In this way, one then correlates the amount or kind of alterations detected with various clinical states and treatment options.

### Cancer Treatments

In some embodiments, the disclosure provides compositions and methods for the diagnosis and treatment of breast cancer. In one embodiment, the disclosure provides a method of determining the treatment of cancer based on whether the level of CTCs is high in comparison to a control. The treatment may be a conventional cancer treatment. One of skill in the art will be aware of many treatments that may be combined with the methods of the present disclosure, some but not all of which are described below.

### A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present disclosure administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the disclosure, methods include identifying a patient in need of treatment A patient may be identified, for example, based on taking a patient history or based on findings on clinical examination.

### B. Treatments

In some embodiments, the method further comprises treating a patient with breast cancer with a conventional cancer treatment. One goal of current cancer research is to find ways to improve the efficacy of chemo- and radiotherapy, such as by combining traditional therapies with other anti-cancer treatments. In the context of the present disclosure, it is contemplated that this treatment could be, but is not limited to, chemotherapeutic, radiation, a polypeptide inducer of apoptosis, a novel targeted therapy such as a tyrosine kinase inhibitor, or an anti-VEGF antibody, or other therapeutic intervention. It also is conceivable that more than one administration of the treatment will be desired.

### 1. Chemotherapy

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2′′-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C″); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

### 2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gene silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance antitumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient’s circulating lymphocytes, or tumor infiltrated lymphocytes, are isolated in vitro, activated by lymphokines such as IL-2 or transduced with genes for tumor necrosis, and re-administered (Rosenberg et al., 1988; 1989).

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs’ surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Gene Therapy

In yet another embodiment, the secondary treatment is a gene therapy in which a therapeutic polynucleotide is administered before, after, or at the same time as a H2A.Z targeting agent is administered. Delivery of a H2A.Z targeting agent in conjunction with a vector encoding one of the following gene products may have a combined anti-hyperproliferative effect on target tissues. A variety of proteins are encompassed within the disclosure, some of which are described below.

### a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present disclosure, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

### b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK’s. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein p16 also is known to regulate the function of CDK6.

p16INK4 belongs to a class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present disclosure include Rb, APC, DCC, NF-1, NF-2, WT-1, MEN-I, MEN-II, zac1, p73, VHL, MMAC1/H2A.Z, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

### c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and the ICE-like proteases have both been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BclXL, BclW, BclS, Mcl-1, A1, Bfl-1) or counteract Bcl-2 function and promote cell death (e.g., Bax, Bak, Bik, Bim, Bid, Bad, Harakiri).

### d. RNA Interference (RNAi)

In certain embodiments, the H2A.Z inhibitor is a double-stranded RNA (dsRNA) directed to an mRNA for H2A.Z.

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). It is generally accepted that RNAi acts post-transcriptionally, targeting RNA transcripts for degradation. It appears that both nuclear and cytoplasmic RNA can be targeted (Bosher and Labouesse, 2000).

### e. siRNA

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA’s guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). siRNA are well known in the art. For example, siRNA and double-stranded RNA have been described in U.S. Pat. Nos. 6,506,559 and 6,573,099, as well as in U.S. Pat. Applications 2003/0051263, 2003/0055020, 2004/0265839, 2002/0168707, 2003/0159161, and 2004/0064842, all of which are herein incorporated by reference in their entirety.

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

### f. Production of Inhibitory Nucleic Acids

dsRNA can be synthesized using well-described methods (Fire et al., 1998). Briefly, sense and antisense RNA are synthesized from DNA templates using T7 polymerase (MEGAscript, Ambion). After the synthesis is complete, the DNA template is digested with DNaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedure may be used to aid this time intensive procedure. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25-mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells (Brummelkamp et al., 2002; Lee et al., 2002; Paul et al., 2002; Sui et al., 2002; Yu et al., 2002). Some of these plasmids are engineered to express shRNAs lacking poly (A) tails (Brummelkamp et al., 2002; Paul et al., 2002; Yu et al., 2002). Transcription of shRNAs is initiated at a polymerase III (pol III) promoter and is believed to be terminated at position 2 of a 4-5-thymine transcription termination site. shRNAs are thought to fold into a stem-loop structure with 3′ UU-overhangs. Subsequently, the ends of these shRNAs are processed, converting the shRNAs into ^{~}21 nt siRNA-like molecules (Brummelkamp et al., 2002). The siRNA-like molecules can, in turn, bring about gene-specific silencing in the transfected mammalian cells.

### g. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL, (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient’s tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient’s organ or a limb is heated for regional therapy, which is accomplished using devices that produce high energy, such as magnets. Alternatively, some of the patient’s blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present disclosure or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### 5. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regard, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### EXAMPLES

### Example 1 Development of a 4-Color Fluorescence In-Situ Hybridization Assay

### Materials and Methods

### Patient Enrollment

Physicians, study subjects, and laboratory and statistical personnel were blinded to the results of the test and clinical information. The blinding protocol was strictly followed, and the results of the test did not direct or influence patient care. All sites had institutional review board approval, and informed written consent was obtained from all eligible participants

Eligible patients are older than 18 years of age scheduled for percutaneous needle biopsy. There are no restrictions on nodule characteristics in order to avoid bias from radiological factors. Patients were ineligible if they had a prior or concurrent cancer diagnosis of any type, or a lung cancer diagnosis within the past two years.

### Blood Collection

Blood was collected just prior to the CT-guided needle biopsy procedure. Blood was collected in vacutainer tubes containing blood stabilizer (Streck, Omaha, Nebraska) and shipped overnight to LungLife AI’s CLIA lab in Thousand Oaks, CA.

### CTC Enrichment

Samples received in the CLIA lab were accessioned into a laboratory information management system using two unique identifiers. Blood was centrifuged at 1000 × g for 10 minutes with the brake off. Plasma was transferred to new tubes and stored at -80° C. Erythrocytes were removed using an ammonium chloride-based erythrocyte lysis buffer. The remaining leukocytes were quantified using a BD Accuri C6 flow cytometer (Becton Dickenson, San Jose, CA) and 5e6 leukocytes were transferred to a new tube for magnetic depletion. Cells were incubated with biotinylated antibodies targeting CD66b and CD14 (BioLegend, San Diego, CA) for removal of neutrophils and monocytes, respectively. This was followed by incubation with paramagnetic streptavidin coated particles (BD Biosciences, San Jose, CA) and subsequent magnetic separation, and the supernatant was transferred to a new tube.

### Cell Cryopreservation and Ampule Thawing

Leukocytes not used in the depletion procedure were washed once with PBS containing 10% FBS. Cells were resuspended in 1 mL cryopreservation medium containing 10% DMSO and slowly frozen in a -80° C. freezer (-1° C./min) and then transferred to liquid nitrogen. Ampules were thawed in a 37° C. water bath for approximately 2 minutes, followed by two washes with 10 mL PBS containing 10% FBS to reduce DMSO.

### Fluorescence In-Situ Hybridization

10,000 to 20,000 cells from the cell suspension were then transferred to a glass slide using a cytospin instrument. Cells were fixed in Carnoy’s fixative (3:1 solution of methanol and glacial acetic acid) for 30 minutes, followed by treatment with protease (pepsin pH=2, Abbot Molecular). 4-color FISH probes (Katz et al. Cancer Cytopathol. 2020;10.10021cncy.22278. doi:10.1002/cncy.22278) were then added to the microscope slide and a coverslip was affixed using rubber cement. DNA was denatured at 80° C. for 2 minutes, followed by overnight hybridization in a humidified chamber for 18 hours. Slides were then washed, and a new coverslip applied with mounting medium containing DAPI (Vector Labs, Burlingame, CA).

### Image Acquisition and Analysis

Slides containing cells were imaged using a Bioview Allegro-Plus microscope system (Bioview USA, Billerica, MA). Images were acquired using a 60x objective (Olympus, UPlanSapo, 1.35 NA oil immersion) and a FLIR Grasshopper 3 monochrome camera (12-bit, 2448 × 2048 pixels, 3.4 µm pixel size) controlled using Bioview Duet software. All cells were imaged with 21 transverse sections spanning 0.65 µm.

Objects were classified by the Bioview Duet software according to probe copy number variation (“normal” cells show 2 spots of each color, “deletion” is a loss of one or more spots, “single-gain” is an extra spot in one color, and “CTC” is defined as a gain in two or more channels). A licensed technician would then analyze cells binned in the “CTC” class by the Bioview Duet software and verify each cell. CTC counts are normalized by dividing the CTC count by the total number of cells analyzed and multiplying by 10,000. A minimum of 10,000 cells are analyzed per subject. Total CTC count, total cell count, and normalized CTC counts were sent for unblinding for each subject.

### Statistics

Receiver-operator characteristics analysis was performed using normalized CTC counts from case and control subjects (malignant and benign nodules, respectively). The statistical significance of clinical factor data was determined using the Mann-Whitney test (two-tailed, 95% confidence interval).

### Results

### CTC Enrichment Optimization

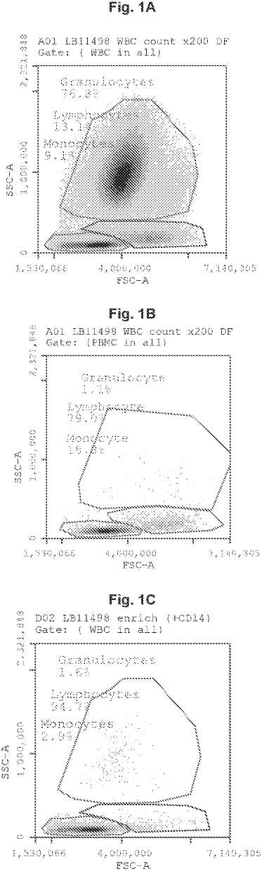

Katz et al described a method of CTC enrichment using Ficoll-based density centrifugation, which mainly removes erythrocytes and granulocytes and leaves peripheral blood monocytes and lymphocytes at the interface layer (Katz et al., Clin Cancer Res. 2010;16(15):3976-3987). While this method has shown high performance (Katz et al. Cancer Cytopathol. 2020;10.1002/cncy.22278. doi:10.1002/cncy.22278), it is technique-driven and limited in throughput required in the clinical setting. Immunomagnetic separation is a simpler process that is highly amendable to automation. Accordingly, the combination of erythrocyte lysis with CD66b-targeted depletion of granulocytes to mirror Ficoll-mediated CTC enrichment was utilized. Flow cytometry shows equivalent removal of erythrocytes and granulocytes using the two methods (FIGS. 1). CTC are identified based on copy number variation and defined as having a gain in two or more channels (FIG. 2). Upon testing in clinical samples of patients with lung cancer lower sensitivity was observed than was previously published. In looking at flow cytometry data pre- and post-enrichment, excessive granulocytes and monocytes were observed in false negative samples (FIG. 3) suggesting that a minimum level of depletion is required to achieve the desired adequate performance level. Additionally, a lower number of total cells scanned in false negative samples was observed compared to true positives, suggesting the number of cells also contributes to performance (FIG. 4). Subsequently, CD14 and CD66b were added to the depletion cocktail in order to remove monocytes and granulocytes, respectively. Using blood from the same patients, the sensitivity was doubled compared to when CD66b alone was used (FIG. 5), suggesting the CTC preferentially co-isolate with the lymphocyte fraction in this assay. The CD14/CD66b cocktail was used throughout the remainder of this study.

### Cryopreservation Effect on Assay Performance

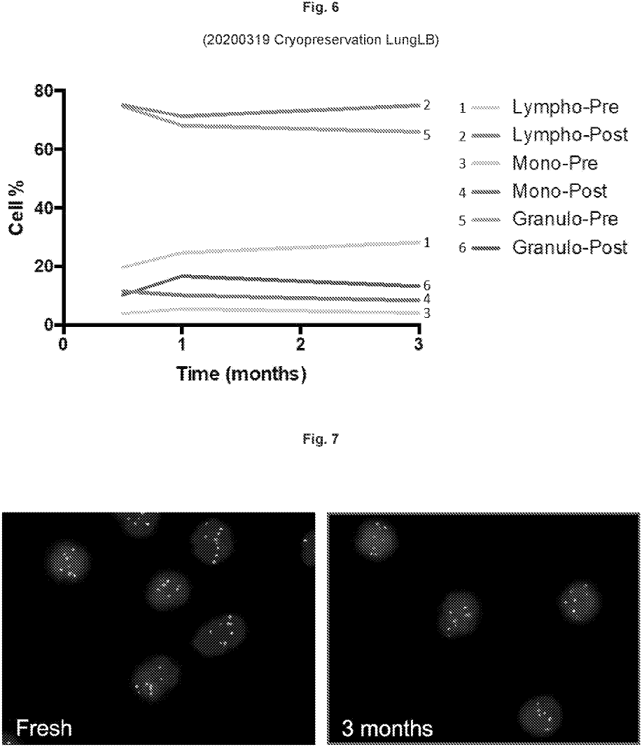

In some aspects, the 4-color fluorescence in-situ hybridization LungLB™ assay requires 5 million cells used as input for the assay, meaning that all plasma and remaining blood cells remain unused and available. While protocols exist for long-term storage of plasma and as such many biobanks are available, there are no known biobanks available for accessing CTC. As such, we attempted multiple protocols to cryoprotect remaining cells which are invaluable for retrospective analysis. Suspension in a solution containing 10% DMSO shows stability at -80° C. for 0.5, 1, 3, and 12 months in terms of efficiency of depletion and FISH (FIG. 6). Stability of cells is depicted in FIG. 7 showing fresh cells and cryopreserved cells following 3 months of cryopreservation.

### Analytical Validation of 4-Color Fluorescence In-Situ Hybridization LungLB™ Assay

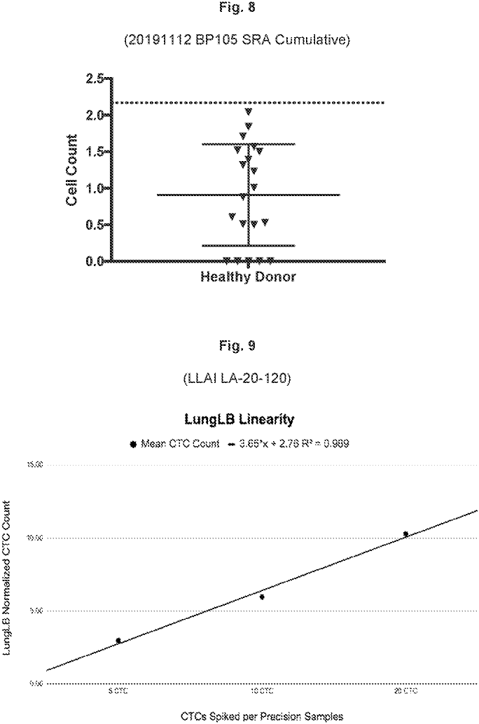

Before commencing the pilot study, the analytical performance of the 4-color fluorescence in-situ hybridization LungLB™ assay was evaluated. First, the 4-color fluorescence in-situ hybridization LungLB™ assay protocol was run on blood samples from 20 unique healthy donors and CTC counts were recorded (FIG. 8). The median CTC count across all samples was 0.945 CTC/10,000 cells analyzed (±0.155 SEM). To understand linearity, A549 lung adenocarcinoma cells were spiked into healthy donor blood at 5, 10, and 20 CTC to represent low, medium, and high adenocarcinoma ranges seen in clinical specimens. The assay performs with strong linearity (R² = 0.989) and has a limit of detection below 5 CTC per 10,000 cells analyzed (FIG. 9), indicating the assay should work across the spectrum of clinical samples.

### Blinded Clinical Sample Analysis

The 4-color fluorescence in-situ hybridization LungLB™ assay is being developed as an aid in the clinical assessment of patients with indeterminate lung nodules. As such, blood samples drawn from 46 subjects at the same time as percutaneous needle biopsy were evaluated. The percutaneous needle biopsy was performed to retrieve sufficient tissue to make a definitive diagnosis on an indeterminate pulmonary nodule. After unblinding, clinical characteristics currently used in malignancy prediction modules were compared in patients with benign versus malignant lesions and no significant differences were found in patient age, smoking history, or nodule size (Table 1), indicating data reflect “real world” scenarios and have no demonstrable selection bias.

TABLE 1

Clinical characteristics in study subjects

| Parameter (mean) | Benign n=15 | Malignant n=31 | P Value |
|---|---|---|---|
| Age (years) | 69.5 | 68.9 | 0.796 |
| Smoking history (pk-yr) | 31.6 | 22.6 | 0.092 |
| Nodule size (cm) | 2.19 | 1.94 | 0.247 |
| Size range (cm) | 0.8 -3.4 | 0.3-4.4 | |
| Nodule Location (n) | | | |
| Right upper lobe | 5 | 10 | |
| Right middle lobe | 0 | 1 | |
| Right lower lobe | 1 | 3 | |
| Left upper lobe | 5 | 10 | |
| Left lower lobe | 4 | 7 | |

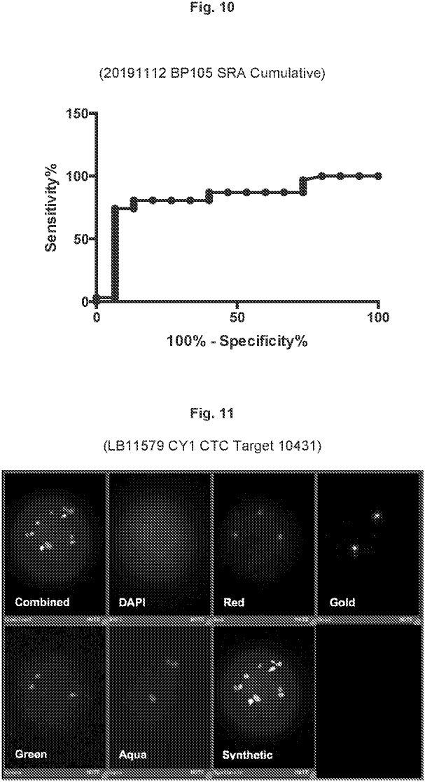

The 4-color fluorescence in-situ hybridization LungLB™ assay demonstrated an area under the receiver operator characteristics (ROC-AUC) curve of 0.823 with a sensitivity of 81% and specificity of 87% at a cutoff of 2.17 CTC/10,000 cells analyzed (FIG. 10). At this cutoff, positive predictive value was calculated to be 92.5% and negative predictive value 68.4%.

One subject of note in the study (LB 11579) was a 64-year-old female former smoker (37 pack per year (pk-yr) smoking history) who presented with a 3 mm suspicious nodule in the left upper lobe. Biopsy was negative for lung cancer; however, the The 4-color fluorescence in-situ hybridization LungLB™ assay returned a positive result (6.87 CTC/10,000), suggestive of a malignant process (FIG. 11). The patient was referred to a thoracic surgeon for a wedge resection and surgical pathology revealed adenocarcinoma.

### Discussion

It is well described that survival rates are higher the earlier lung cancer is detected. This is reflected in both the NLST (Aberle et al. (2011) N Engl J Med 365(5): 395-409) and NELSON (de Koning HJ, van der Aalst CM, de Jong PA, et al. Reduced Lung-Cancer Mortality with Volume CT Screening in a Randomized Trial. N EnglJ Med. 2020;382(6):503-513) trials on lung cancer screening. However, non-malignant pulmonary nodules are very common, and the false discovery rate using LDCT has been reported to be over 95% (Aberle et al. (2011) N Engl J Med 365(5): 395-409). The sub-optimal specificity of imaging, along with the deficiencies in the shared decision-making process required for lung cancer screening, contributes to the low uptake of lung cancer screening for those deemed at risk (Jemel and Fedawa, JAMA Oncol. 2017;3(9):1278-1281). In fact, clinicians report having fewer conversations with at-risk patients about lung cancer screening year-over-year, likely due in part to the lack of an effective screening solution (Huo et al. Cancer Epidemiol Biomarkers Prev. 2019;28(5):963-973.). A non-invasive tool capable of providing additional information in the context of the indeterminate pulmonary nodule is needed.

Emerging technologies for noninvasive early detection of lung cancer detect analytes in blood, including CTC, circulating tumor DNA (ctDNA), and immune response markers (Seijo et al. (2019) J Thorac Oncol 14(3): 343-357). Of these, CTC are perhaps the most sensitive and specific markers of early lung cancer, with the fewest technical limitations. ctDNA, while abundant in late-stage lung cancer due to a general abundance of necrotic and apoptotic lesions, is limiting in early stage disease when tumors are small and the greatest benefit from curative surgery can be obtained, and thus sensitivity using ctDNA is insufficient for clinical decision making (Abbosh et al. (2018) Nat Rev Clin Oncol. 15(9):577-586). While the immune response has evolved to detect and respond to malignancy, current molecular and mechanistic knowledge is limiting. For example, autoantibodies detecting tumor neoantigens have been deployed to detect the presence of malignancy; however, sensitivity of these assays is likely low because these neoantigens do not cover the spectrum of lung cancers. Additionally, because pulmonary nodules can be formed by many immune-responsive insults such as fungal and viral infections, a peripheral immune-response or field-effect-type approach can be challenged by the heterogeneity of benign lesions. CTC, on the other hand, represent an appropriate analyte as they take advantage of an evolutionarily conserved biological process in the lung. Lung cells have a high propensity for motility which is observed in vivo following damage to the lung epithelium (Vaughan et al. (2015) Nature 517(7536):621-625, Kathiriya et al. (2020) Cell Stem Cell. 26(3):346-358), and it is likely this mechanism has been conserved during malignant transformation. By defining CTC based on copy number variation using DNA FISH, which is indelible (i.e. DNA), minimizes influences from transcriptional or translational changes in the cell.

Using a CTC-based liquid biopsy, the 4-color fluorescence in-situ hybridization LungLB™ assay described herein is capable of discriminating benign from malignant processes in subjects with indeterminate pulmonary nodules at risk for lung cancer. This assay performs with both high sensitivity and specificity because 1) it utilizes CTC which are found at early stages of lung cancer pathogenesis and 2) uses DNA copy number variation via FISH as a readout, which in general is a highly specific assay.

### Example 2 Effects of Sodium Bicarbonate Concentration on Granulocyte Size

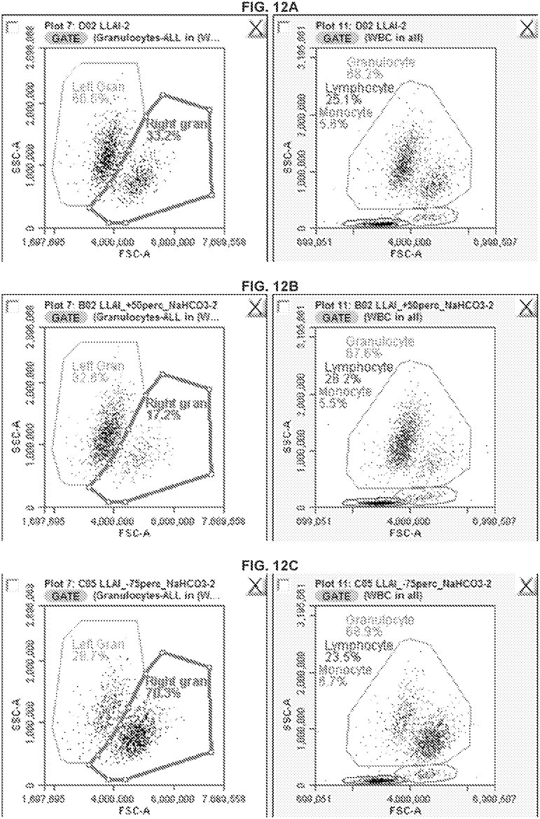

FIG. 12A depicts a standard lysis buffer where ~66% of the granulocytes shifted to be smaller in size. A 50% increase in sodium bicarbonate concentration resulted in~82% of granulocytes shifting to be smaller in size), as shown in the middle panel (FIG. 12B). FIG. 12C depicts a reduction in granulocyte shrinkage in a lysis buffer with a 75% decrease in sodium bicarbonate concentration.

### Example 3: Three Antibody CTC Enrichment Method

The effects of adding one or more additional antibodies to the depletion cocktail was assessed. A depletion cocktail containing CD66b, CD14,and CD3 antibodies was evaluated.

Results from the ImmunoFISH study determined LungLB target cells are either CD45+/CD3- or CD45-/CD3- suggesting that target cells could be both certain immune cells or classic epithelial CTC’s. Both populations of CTCs are CD3 Negative, presenting the opportunity to further enrich LungLB samples by adding a biotinylated-CD3 antibody to the depletion cocktail. The LungLB v2 cocktail includes CD66b and CD14 biotinylated antibodies. LungLB v3 includes biotinylated CD66b, CD14, and CD3 antibodies.

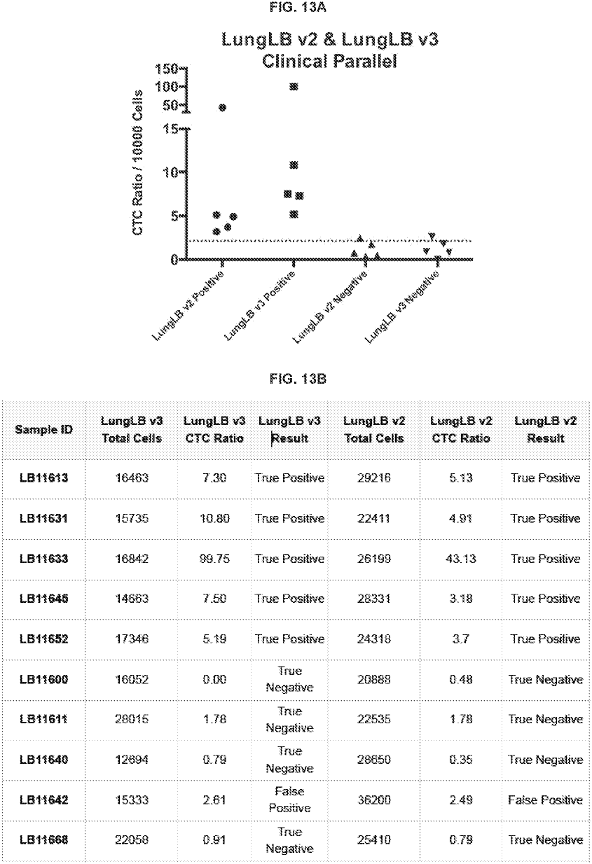

True Positive clinical samples processed with the LungLB v3 assay produced on average twice as many CTC’s compared to LungLB v2 (FIGS. 13A and 13B). This study was performed across 5 unique lung cancer positive patients ranging from Stage I to Stage IV including Adenocarcinoma, Squamous Cell Carcinoma, SCLC, & Neuroendocrine Carcinoma. This provides a high level of confidence that LungLB CTC’s are CD3 Negative. A true positive sample indicates a patient with malignant lung cancer. A true negative sample indicates a patient with a benign lung nodule. A false positive sample indicates a positive result for a patient having a benign lung nodule. A false negative sample indicates a negative result where a patient has malignant lung cancer.

### Example 4: LungLB Assay and CD45 Immunostaining

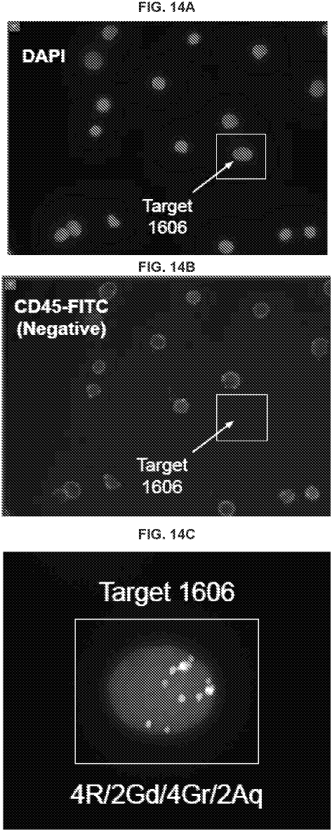

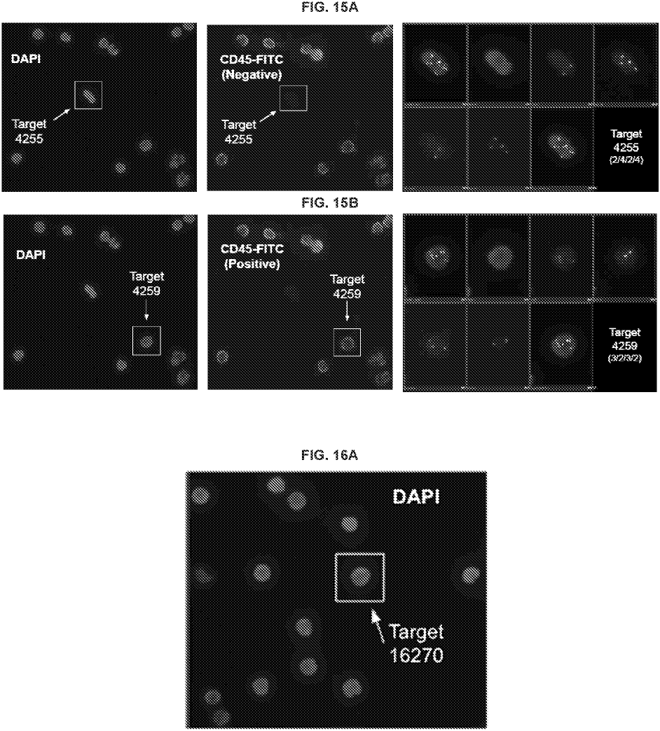

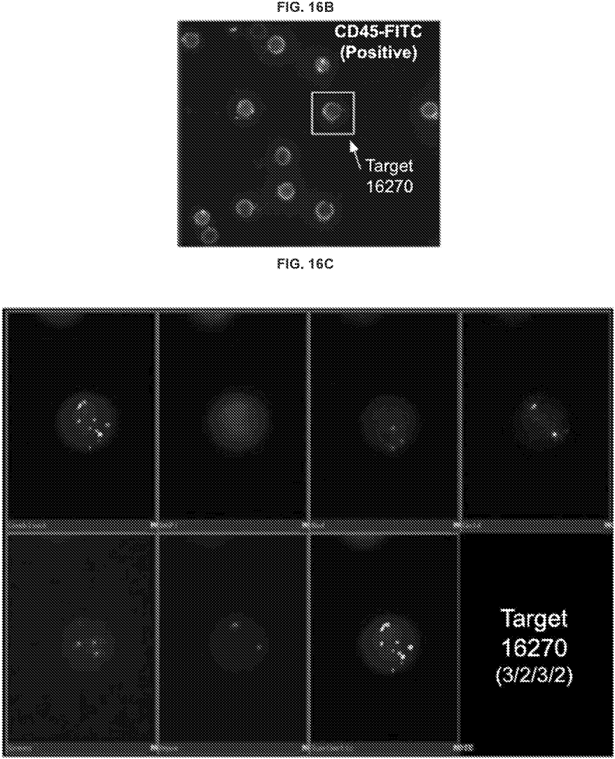

ImmunoFISH has been used in R&D settings to determine surface markers present on LungLB CTCs. CD45 is a commonly used surface marker to differentiate epithelial CTCs from hematopoietic White Blood Cells. While most cells in FIGS. 14A and 14B are CD45 positive, the advanced CTC with a probe pattern of 4R/2Gd/4Gr/2Aq is CD45 negative.

Multiple Double Deletion CTCs and 4×2 CTCs have been discovered to be CD45 Negative in previous ImmunoFISH studies.

CD45+ target cells generally present a 3R/2Gd/3Gr/2Aq probe pattern and are observed in both malignant and benign patient samples at varying degrees. CD45- target cells generally present more advanced probe patterns such as 5R/lGd/5Gr/lAq (Double Deletion) or 2R/4Gd/2Gr/4Aq (4×2 CTC). These target cells have significantly higher specificity for lung cancer compared to the CD45+ target cells.

### Example 5: CTC Enrichment Utilizing Anti-CD19 and Anti-CD56 Antibodies

The use of additional biomarkers and antibodies that may be used to further enrich samples and increase the number of CTCs in the LungLB assay. Potential additional antibodies to be tested include CD3 (T-Cells), CD19 (B-Cells) & CD56 (NK-Cells).

LungLB results are identified as negative or positive based on an established threshold of CTCs per ten thousand total cells. A LungLB Positive result suggests the sample is from a patient with malignant lung cancer. A negative LungLB result suggests the sample is from a patient with a benign nodule.

### RESULTS

The starting percentage of leukocyte subpopulations in patient LB11697 provides a baseline necessary to assess enrichment efficiency in the final samples Table 2 lists the starting white blood cell (WBC) composition of the patient sample.

TABLE 2

Starting Percentage of WBC Populations

| Sample ID | Starting % Granulocyte | Starting % Monocyte | Starting % Lymphocyte | Starting % CD3+T-Cell | Starting % CD19+ B-Cell | Starting % CD56+NK Cell |
|---|---|---|---|---|---|---|
| LB11697 | 67.6 | 6.8 | 24.7 | 81.5 | 9.1 | 7.0 |

Table 3 depicts the enriched percentage of leukocyte subpopulations when processed with various antibody cocktails using CD66b, CD14, CD3, CD19, or CD56. LungLB v4.1 using an anti-CD19 antibody in addition to anti-CD66b, anti-CD14 and anti-CD3 antibodies reduced the percentage of B-Cells down to 0.1% and enriched NK-Cells to 72.9%. LungLB v4.2 using an anti-CD56 antibody in addition to anti-CD66b, anti-CD14, and anti-CD3 antibodies reduced the percentage of NK-Cells down to 2.2% and enriched B-Cells to 70.5%. LungLB v4.3 using an anti-CD56 antibody and an anti-CD19 antibody in addition to anti-CD66b, anti-CD14, and anti-CD3 antibodies reduced the percentage of NK-Cells down to 9.2% and reduced B-Cells to 0.2%.

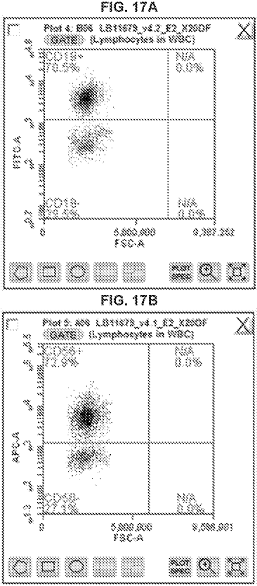

Samples were processed with Flow Cytometry by staining the enriched cell populations with the immunofluorescent versions of the depletion antibodies. This is an orthogonal method used to confirm exactly what percentage of leukocyte subpopulations are on each slide before they are processed with FISH (FIGS. 17A and 17B)

TABLE 3

Enriched Percentage of WBC Populations

| Antibody Cocktail | Enriched % Granulocyte | Enriched % Monocyte | Enriched % Lymphocyte | Enriched % CD3+ T-Cell | Enriched % CD19+ B-Cell | Enriched % CD56+ NK Cell |
|---|---|---|---|---|---|---|
| LungLB v2 (CD66b / CD14) | 13.6 | 2.4 | 83.6 | 81.5 | 9.1 | 7.0 |
| LungLB v3 (CD66b / CD14 / CD3) | 15.0 | 3.8 | 80.5 | 11.0 | N/A | N/A |
| LungLB v4.1 (CD66b / CD14 / CD3 / CD19) | 7.5 | 3.0 | 87.2 | 0.7 | 0.1 | 72.9 |
| LungLB v4.2 (CD66b / CD14 CD3 / CD56) | 10.8 | 2.6 | 84.4 | 0.5 | 70.5 | 2.2 |
| LungLB v4.3 (CD66b / CD14 / CD3 / CD19 / CD56) | 19.2 | 7.0 | 70.2 | 2.2 | 0.2 | 9.2 |

Antibody Cocktails with CD19 added (B-Cell Depletion) drastically reduced the overall number of CTCs observed in clinical sample LB11679 (Table 4). This suggests that B-Cells comprise a large majority of target cells. As samples were further enriched the number of Advanced CTC Subtypes (Double Deletions) was maintained and even increased noticeably in the LungLB v4.3 cocktail with all 5 antibodies. B-Cells may be necessary in early lung cancer diagnosis. The Advanced CTC Subtypes that continue to enrich even with all 5 depletion antibodies may be bona fide tumor cells. Positive selection of CD19+ B-cells can offer further diagnostic advantages including...

Analyzing CD45+/CD19+ target B-Cells separately from the remaining enriched cells containing true CTCs provides the opportunity to produce a more accurate lung cancer diagnosis by attacking the problem from two pathways.

Removing B-Cells from the final enriched sample leaves only true CD45- epithelial CTCs remaining, as supported by the LungLB v4.3 advanced CTC numbers. This should drastically increase assay specificity. However, assay sensitivity may decrease because these true CD45- epithelial CTCs remaining are difficult to enrich and may not be present in every patient.

Abnormal B-Cells are observed in both malignant and benign patients, even normal healthy donors, to varying degrees. While abnormal CD45+ B-Cells might not be less specific to lung cancer as the CD45- CTCs, CD45+ B-Cells increase assay sensitivity and enable early detection.

TABLE 4

LungLB FISH Results / CTC Counts

| Sample ID | Antibody Cocktail | Total Cells Scanned | CTC Count | Normalized CTC Ratio | Advanced CTC Subtypes |
|---|---|---|---|---|---|
| LB11679 | LungLB v2 (CD66b / CD14) | 31476 | 24 | 7.63 | Double Deletion: 0 4×2 CTC: 0 |
| LB11679 | LungLB v3 (CD66b / CD14 / CD3) | 29979 | 68 | 22.68 | Double Deletion: 0 4×2 CTC: 1 |
| LB11679 | LungLB v4.1 (CD66b / CD14 / CD3 / CD19) | 29996 | 4 | 1.33 | Double Deletion: 1 4×2 CTC: 1 |
| LB11679 | LungLB v4.2 (CD66b / CD14 / CD3 / CD56) | 29986 | 124 | 41.35 | Double Deletion: 1 4×2 CTC: 0 |
| LB11679 | LungLB v4.3 (CD66b / CD14 / CD3 / CD19 / CD56) | 29988 | 9 | 3.00 | Double Deletion: 3 4×2 CTC: 2 |

1. A method for identifying a subject at risk for the development of lung cancer comprising:
(a) obtaining a test sample from a human subject;
(b) performing a circulating tumor cell (CTC) enrichment step comprising:
(i) removing plasma from the sample,
(ii) removing erythrocytes from the sample,
(iii) contacting the sample with at least one biotinylated affinity agent that binds a cell surface marker, and
(iv) contacting the sample with streptavidin-coated magnetic particles and depleting cells from the sample that express the cell surface marker;
(c) hybridizing the enriched cells in the sample with labeled nucleic acid probes that hybridize to regions of chromosomal DNA;
(d) evaluating the signal pattern for the selected cells by detecting fluorescence in situ hybridization from cells;
(e) detecting CTCs based on the pattern of hybridization to the labeled nucleic acid probes to said selected cells; and
(f) identifying the subject at risk for the development of lung cancer when the number of CTC per sample is above a predetermined cutoff value.

2. The method of claim 1, wherein the test sample is blood.

3. The method of claim 1, wherein the erythrocytes are removed by cell lysis.

4. The method of claim 3, wherein the cell lysis is performed by an ammonium chloride lysis buffer.

5. The method of claim 1, wherein the plasma is removed by centrifugation.

6. The method of claim 1, wherein the cell surface marker is selected from CD66b, CD14, CD3, CD4, CD8, CD17, CD56, CD19, CD20, CD25, IgM, or IgD.

7. The method of claim 1, wherein the cell surface marker is selected from CD66b, CD3 or CD14.

8. The method of claim 1, wherein the cell surface marker comprises CD66b and CD14.

9. The method of claim 1, wherein the cell surface marker comprises CD66b, CD14 and CD3.

10. The method of claim 1, wherein the cell surface marker comprises CD66b, CD14, CD3, and CD56.

11. The method of claim 1, wherein the cell surface marker comprises CD66b, CD14, CD3, and CD 19.

12. The method of claim 1, wherein the cell surface marker comprises CD66b, CD14, CD3, CD56 and CD19.

13. The method of claim 1, wherein the at least one biotinylated affinity agent comprises an anti-CD66b, anti-CD3, anti-CD56, anti-CD19 or anti-CD14 antibody.

14. The method of claim 13, wherein the at least one biotinylated affinity agent comprises an anti-CD66b antibody and an anti-CD14 antibody.

15. The method of claim 13, wherein the at least one biotinylated affinity agent comprises an anti-CD66b antibody, an anti-CD14 antibody, and an anti-CD3 antibody.

16. The method of claim 13, wherein the at least one biotinylated affinity agent comprises an anti-CD66b antibody, an anti-CD14 antibody, an anti-CD3 antibody, and an anti-CD56 antibody.

17. The method of claim 13, wherein the at least one biotinylated affinity agent comprises an anti-CD66b antibody, an anti-CD14 antibody, an anti-CD3 antibody, and an anti-CD19 antibody.

18. The method of claim 13, wherein the at least one biotinylated affinity agent comprises an anti-CD66b antibody, an anti-CD14 antibody, an anti-CD3 antibody, an anti-CD56 antibody, and an anti-CD19 antibody.

19. The method of claim 1, wherein the depleted cells are neutrophils, monocytes, or lymphocytes.

20. The method of claim 1, wherein the depleted cells are neutrophils and monocytes.

21. The method of claim 1, wherein the CTC enrichment step further comprises:
(i) contacting the sample with at least one additional biotinylated affinity agent that binds a cell surface marker, and
(iv) contacting the sample with streptavidin-coated magnetic particles and collecting cells that express the cell surface marker.

22. The method of claim 21, wherein the cell surface marker comprises at least one of CD19, CD20, IgM, or IgD.

23. The method of claim 21, wherein the at least one additional biotinylated affinity agent comprises at least one of an anti-CD19 antibody, an anti-CD20 antibody, an anti-IgM antibody, or an anti-igD antibody.

24. The method of claim 21, wherein the collected cells comprise lymphocytes.

25. The method of claim 24, wherein the lymphocytes are B-cells.

26. The method of claim 1, wherein the labeled nucleic acid probes comprise 3p22.1, 10q22.3, chromosome 10 centromeric (cep10), and 3q29.

27. The method of claim 1, wherein the subject at risk has indeterminate pulmonary nodules.

28. The method of claim 1, wherein a CTC is identified when the hybridization pattern of the nucleic acid probes depicts a gain of two or more chromosomal regions in a cell.

29. The method of claim 1, wherein a CTC is identified when the hybridization pattern of the nucleic acid probes depicts a loss of two or more chromosomal regions in a cell.

30. The method of claim 1, wherein a CTC count greater than 1 CTC/10,000 cells represents a risk of lung cancer.

31. The method of claim 1, wherein a CTC count greater than 2 CTC/10,000 cells represents a risk of lung cancer.

32. The method of claim 1, wherein a CTC count greater than 2.5 CTC/10,000 cells represents a risk of lung cancer.

33. The method of claim 1, wherein a CTC count greater than 5 CTC/10,000 cells represents a risk of lung cancer.

34. The method of claim 1, wherein a CTC count greater than 10 CTC/10,000 cells represents a risk of lung cancer.

35. The method of claim 1, wherein a CTC count greater than 20 CTC/10,000 cells represents a risk of lung cancer.

36. The method of claim 1, wherein the subject with a CTC count greater than 5 CTC/10,000 cells is referred for surgical resection of the nodule.

37. The method of claim 1, wherein the labeled nucleic acid probes for 3p22.1 is an RPL14, CD39L3, PMGM, or GC20 probe.

38. The method of claim 1, wherein the labeled nucleic acid probes for 10q22.3 is a surfactant protein A1 or surfactant protein A2 probe.

39. A method for identifying a subject at risk for the development of lung cancer comprising:
(a) obtaining a test sample from a human subject;
(b) performing a circulating tumor cell (CTC) enrichment step comprising:
(i) removing plasma from the sample,
(ii) removing erythrocytes from the sample,
(iii) contacting the sample with at least one biotinylated affinity agent that binds a cell surface marker, and
(iv) contacting the sample with streptavidin-coated magnetic particles and collecting cells from the sample that express the cell surface marker;
(c) hybridizing the enriched cells in the sample with labeled nucleic acid probes that hybridize to regions of chromosomal DNA;
(d) evaluating the signal pattern for the selected cells by detecting fluorescence in situ hybridization from cells;
(e) detecting CTCs based on the pattern of hybridization to the labeled nucleic acid probes to said selected cells; and
(f) identifying the subject at risk for the development of lung cancer when the number of CTC per sample is above a predetermined cutoff value.

40. The method of claim 39, wherein the cell surface marker is selected from CD66b, CD14, CD3, CD4, CD8, CD17, CD56, CD19, CD20, CD25, IgM, or IgD.

41. The method of claim 39, wherein the cell surface marker is a B-cell specific cell surface marker.

42. The method of claim 41, wherein the B-cell specific cell surface marker is CD19, CD20, IgM, or IgD.

43. The method of claim 42, wherein the at least one biotinylated affinity agent comprises an anti-CD19 antibody, an anti-CD20 antibody, an anti-IgM antibody, or an anti-IgD antibody.

44. A method of evaluating cancer in a subject comprising determining the level of circulating tumor cells (CTCs) in a sample containing blood cells from the patient by the method of any one of the preceding claims, wherein a higher level of CTCs in the sample, as compared to a control or predetermined number of CTCs from a non-aggressive form of cancer, is indicative of an aggressive form of cancer and/or a poor cancer prognosis.

45. A method of staging cancer in a subject comprising determining circulating tumor cells (CTC) in a sample containing blood cells from the subject by the method of any one of the preceding claims, wherein a higher level of CTCs in the sample as compared to a predetermined control for a given stage is indicative of a more advanced stage of cancer, and a lower level of CTCs in the sample as compared to a control for a given stage is indicative of a less advanced stage of cancer.
